(19) United States
(12) Patent Application Publication
(10) Pub. No.: US 2012/0295346 A1
(43) Pub. Date: Nov. 22, 2012

(54) METHODS AND COMPOSITIONS FOR MODULATING MEMBRANE POTENTIAL TO INFLUENCE CELL BEHAVIOR

(75) Inventor: Michael Levin, Swampscott, MA (US)

(73) Assignee: Tufts University, Boston, MA (US)

(21) Appl. No.: 13/386,372

(22) PCT Filed: Jul. 22, 2010
(86) PCT No.: PCT/US2010/042952

§ 371 Date: Aug. 7, 2012

Related U.S. Application Data
(60) Provisional application No. 61/227,708, filed on Jul. 22, 2009.

(51) Int. Cl. C12N 5/02; C12N 5/0735; C12N 5/0775

(52) U.S. Cl. 435/366; 435/375

(57) ABSTRACT

The present invention provides methods for controlling proliferation, differentiation, and/or migration of cells by modulating membrane potential through an endogenously expressed channel protein. The present invention also provides methods for identifying candidate instructor cells, as well as therapeutic agents that are useful for modulating (e.g., promote or inhibit) proliferation and differentiation, as well as promoting regeneration of a desired tissue type.

### RELATED APPLICATIONS

This application claims the benefit of priority to U.S. provisional application No. 61/227,708, filed Jul. 22, 2009, the disclosure of which is hereby incorporated by reference in its entirety.

### BACKGROUND OF THE INVENTION

The regeneration of complex tissues and organ systems lost to injury, senescence, or disease is a key goal of biomedicine. In addition to its clinical applications, the regeneration of organs is fascinating because it represents one of the most fundamental properties of most living things: recognition of damage and self-repair. The process of regeneration, embryonic development and neoplasm undoubtedly result from a complex orchestration of growth and morphogenesis. Currently, substantial efforts are being invested in academia and industry to understand the fundamental principles influencing differentiation and regeneration so that the powers of these processes can be harnessed and used to treat degenerative diseases and injuries.

### SUMMARY OF THE INVENTION

The present invention provides methods for controlling proliferation, differentiation, and/or migration of cells by modulating membrane potential.

In one aspect, the invention provides a method of promoting tissue regeneration, comprising contacting an effective amount of a macrocyclic lactone to a cell culture, wherein the macrocyclic lactone alters the membrane potential of said cells through an endogenous ligand-gated channel expressed in said cell, thereby promoting tissue regeneration. For example, the macrocyclic lactone may be added to the cell culture media so as to act on the cells. In certain embodiments, promoting tissue regeneration comprises promoting one or more of proliferation, differentiation, migration, or survival.

In another aspect, the invention provides the use of a macrocyclic lactone for promoting tissue regeneration, comprising contacting an effective amount of said macrocyclic lactone to a cell culture, wherein the macrocyclic lactone alters the membrane potential of said cells through an endogenous ligand-gated channel expressed in said cell, thereby promoting tissue regeneration.

In certain embodiments, the cell culture of any of the foregoing aspects comprises progenitor cells. In related embodiments said progenitor cell is a neural crest progenitor cell, a mesenchymal stem cells, or a human mesenchymal stem cell. In other related embodiments, said progenitor cell is a neural stem cell or a neuroectodermal stem cell. In some embodiments, said progenitor cell is an embryonic stem cell.

In any of the foregoing aspects and/or embodiments, said macrocyclic lactone is selected from at least one member of the group: avermectin, ivermectin, eprinomectin, abamectin, or moxidectin.

In any of the foregoing aspects and/or embodiments, said use or method promotes proliferation, differentiation, and/or migration of the progenitor cell. In certain embodiments, said progenitor cell becomes depolarized. In other embodiments, said progenitor cell becomes hyperpolarized.

In any of the foregoing aspects and/or embodiments, said ligand-gated channel is a chloride channel. For example, the chloride channel may be GlyCl, or a GABA(A) channel.

In any of the foregoing aspects and/or embodiments, promoting tissue regeneration comprises promoting proliferation, differentiation, and/or migration of one or more of epithelial tissue, muscle tissue, nervous tissue, or connective tissue.

In certain embodiments, any of the foregoing aspects and/or embodiments further comprise decreasing the extracellular chloride concentration relative to that in the progenitor cell to promote efflux of chloride ions from the progenitor cell, thereby altering the membrane potential of the progenitor cell. In other embodiments, the foregoing aspects and/or embodiments further comprise increasing the extracellular chloride concentration relative to that in the progenitor cell to promote influx of chloride ions to the progenitor cell, thereby altering the membrane potential of the progenitor cell.

In one aspect, the invention provides use of a macrocyclic lactone for inhibiting proliferation, differentiation and/or migration of a depolarized cell, wherein said macrocyclic lactone restores the depolarized state of the cell, thereby inhibiting proliferation, differentiation and/or migration. In certain embodiments, this use comprises the use of a macrocyclic lactone for treating cancer. In certain embodiments, the cancer treated is melanoma. In other embodiments, the cancer is a cancer of a neural crest derivative other than melanocytes.

In another aspect, the invention provides use of a selective serotonin reuptake inhibitor (SSRI) for inhibiting proliferation, differentiation and/or migration of a depolarized cell, wherein said SSRI restores the depolarized state of the cell, thereby inhibiting proliferation, differentiation and/or migration. In certain embodiments, the SSRI is a fluoxetine. In other embodiments, the SSRI is not fluoxetine. In certain embodiments, this use comprises the use of an SSRI for treating cancer. In certain embodiments, the cancer treated is melanoma. In other embodiments, the cancer is a cancer of a neural crest derivative other than melanocytes.

In some embodiments, said cell in any of the foregoing aspects is a cancer cell, such as, for example, a melanoma cell. In certain embodiments, said cancer cell is a melanoma cell.

In other embodiments, said cell in any of the foregoing aspects is a neural crest derivative such as, for example, melanocytes or melanocyte precursors.

In certain embodiments, said cell in any of the foregoing aspects is a progenitor cell such as, for example, a mesenchymal stem cell or, more specifically, a human mesenchymal stem cell. In some embodiments, said progenitor cell is a neural stem cell or a neuroectodermal stem cell. In other embodiments, said progenitor cell is an embryonic stem cell.

In some embodiments, said macrocyclic lactone in any of the foregoing aspects and embodiments is selected from at least one member of the group: avermectin, ivermectin, eprinomectin, abamectin, or moxidectin.

In one aspect, the invention provides a method of promoting tissue regeneration, comprising administering an effective amount of an agent to alter the membrane potential of an instructor cell, wherein said agent alters the membrane potential of the instructor cell through an endogenous ligand-gated channel expressed in said instructor cell, thereby promoting tissue regeneration. In certain embodiments, altering the membrane potential of the instructor cell modulates proliferation, differentiation, and/or migration of one or more other cells which are responsive to the instructor cell (e.g., responder cells). Such responder cell or cells can be a progenitor cell, a partially differentiated cell, or a terminally differentiated cell. Moreover, such responder cell or cells can be located at a distance from the instructor cell. In certain embodiments, altering the membrane potential of the instructor cell modulates proliferation, differentiation, and/or migration of one or more neural crest derivatives or one or more neural or neuroectodermal derivatives. In some embodiments, the one or more neural crest derivatives are melanocytes.

In certain aspects, the invention also provides a method of promoting one or more of proliferation, differentiation, and/or survival of a cell, comprising administering an effective amount of an agent to alter the membrane potential of an instructor cell, wherein said agent alters the membrane potential of the instructor cell through an endogenous ligand-gated channel expressed in said instructor cell, thereby promoting proliferation, differentiation, and/or survival of one or more cells modulated by said instructor cell. Such responder cell or cells (e.g., one or more cells modulated by the instructor cell) can be a progenitor cell, a partially differentiated cell, or a terminally differentiated cell. Moreover, such responder cell or cells can be located at a distance from the instructor cell.

In a related embodiment, any of the foregoing methods may further comprise decreasing the extracellular ionic concentration relative to that in the instructor cell to promote efflux of ions from the instructor cell, thereby altering the membrane potential of the instructor cell. In another embodiment, the method may further comprise increasing the extracellular ionic concentration relative to that in the instructor cell to promote influx of ions to the instructor cell, thereby altering the membrane potential of the instructor cell. In either embodiment, the instructor cell may be depolarized or hyperpolarized.

In certain embodiments, the extracellular ionic concentration may be an extracellular chloride concentration, wherein the extracellular chloride concentration is decreased to promote efflux of chloride ions from the instructor cell, thereby depolarizing the instructor cell. In another embodiment, the extracellular ionic concentration may be an extracellular chloride concentration, wherein the extracellular chloride concentration is increased to promote influx of chloride ions to the instructor cell, thereby hyperpolarizing the instructor cell.

In some embodiments, depolarizing the membrane potential of the instructor cell promotes proliferation, differentiation, and/or migration of melanocytes or melanocyte precursors. For example, depolarizing the membrane potential of the instructor cell causes hyperpigmentation due to an increase in proliferation, differentiation, and/or migration of melanocytes or melanocyte precursors. In some embodiments, the ligand-gated channel is a chloride channel, e.g., a glycine-gated chloride (GlyCl) channel, or a GABA(A) channel. In certain embodiments, a small molecule may be used to modulate the ligand-gated channel. By way of example, the small molecule may be a macrocyclic lactone selected from at least one member of the group: avermectin, ivermectin, eprinomectin, abamectin, or moxidectin. In other embodiments, the small molecule may be muscimol. In certain embodiments, the ligand-gated channel is a K_{ATP} channel, e.g., Kir6.1- or Kir6.2-type subunits with sulfonylurea receptors (SUR). By way of example, small molecules such as diazoxide, pinacidil, or glibenclamide can be used as modulators of K_{ATP} channels.

In other embodiments, promoting tissue regeneration may comprise promoting proliferation, differentiation, and/or migration of one or more of epithelial tissue, muscle tissue, nervous tissue, or connective tissue. In some embodiments, the method of promoting one or more of proliferation, differentiation, and/or survival of a cell may comprise promoting proliferation, differentiation, and/or migration of one or more of epithelial tissue, muscle tissue, nervous tissue, or connective tissue. In any of the foregoing embodiments, the instructor cell need not be in direct contact with the tissue for which tissue regeneration is promoted. In other words, the instructor cell acts, at least in part, in a cell non-autonomous fashion to influence directly or indirect a separate population of cells (e.g., responder cells). In a particular embodiment, the instructor cell may be or may influence a human mesenchymal stem cell.

In another aspect, the invention provides a method for modulating proliferation, differentiation and/or migration of one or more neural crest derivative cells or one or more neural derivative cells, comprising: i) identifying an instructor cell capable of modulating proliferation, differentiation, and/or migration of said neural crest derivative cells or said neural derivative cells, ii) identifying one or more ligand-gated channels endogenously expressed in the instructor cell, and iii) administering an effective amount of an agent to alter membrane potential of the instructor cell through the ligand-gated channel, whereby said instructor cell modulates proliferation, differentiation, and/or migration of one or more neural crest derivative cells or one or more neural derivative cells.

In a related embodiment, the method may further comprise decreasing the extracellular ionic concentration relative to the instructor cell to promote efflux of ions from the instructor cell, thereby altering the membrane potential of the instructor cell. In another related embodiment, the method may further comprise increasing the extracellular ionic concentration relative to the instructor cell to promote influx of ions to the instructor cell, thereby altering the membrane potential of the instructor cell.

In any of the foregoing embodiments, the one or more neural crest derivatives may be melanocytes. In certain embodiments, the one or more ligand-gated channels may be, e.g., a chloride channel or a potassium channel. In one embodiment, the chloride channel may be, e.g., a glycine-gated chloride (GlyCl) channel, or a GABA(A) channel. In some embodiments, the agent that modulates the ligand-gated channel may be a small molecule such as a macrocyclic lactone selected from one or more of the group: avermectin, ivermectin, eprinomectin, abamectin, or moxidectin. In other embodiments, the small molecule may be muscimol.

Another aspect of the invention provides a method for identifying a population of instructor cells that modulate proliferation, differentiation and/or migration of a neural crest derivative, comprising contacting a sample with an agent that detects expression of a glycine-gated chloride (GlyCl) channel, wherein cells in the sample that express the glycine-gated chloride (GlyCl) channel are identified as instructor cells that modulate proliferation, differentiation and/or migration of a neural crest derivative. In some embodiments, the neural crest derivative are melanocytes.

In certain aspects, the invention provides a method for identifying an instructor cell capable of modulating proliferation, differentiation, and/or migration, comprising: i) identifying one or more ligand-gated channels endogenously expressed by a discrete population of candidate instructor cells, ii) administering an effective amount of an agent to alter membrane potential of the candidate instructor cell through the identified ligand-gated channel, and iii) assaying for the presence or absence of a desired phenotype, whereby the presence or absence of a desired phenotype indicates that the candidate instructor cell is an instructor cell capable of modulating proliferation, differentiation, and/or migration of one or more other cells.

In related embodiments, any of the foregoing methods may comprise detecting mRNA expression of the glycine-gated (GlyCl) channel. In another embodiment, the method may comprise detecting protein expression of the glycine-gated (GlyCl) channel. In other embodiments, the agent may be an antisense probe that hybridizes to a nucleic acid encoding the GlyCl channel.

In another aspect, the invention provides a method of promoting proliferation, differentiation, and/or survival of a cell. The method comprises administering an effective amount of an agent to alter the membrane potential of an instructor cell, wherein said agent alters the membrane potential of the instructor cell through an endogenous ligand-gated channel expressed in said instructor cell, thereby promoting proliferation, differentiation, and/or survival of one or more cells modulated by said instructor cell. In certain embodiments, altering the membrane potential of the instructor cell modulates proliferation, differentiation, and/or migration of one or more neural crest derivatives or one or more neural derivatives. In some embodiments, the one or more neural crest derivatives are melanocytes.

In other aspects, the invention provides a method of modulating proliferation, differentiation, and/or migration of a subject cell, wherein the subject cell expresses a GlyCl channel, comprising administering to the subject cell an effective amount of a GlyCl channel modulator to alter the membrane potential of the subject cell, thereby modulating the proliferation, differentiation, and/or migration of the subject cell.

In some embodiments, the modulator may be a small molecule. In certain embodiments, the GlyCl channel modulator may be a macrocyclic lactone, e.g., avermectin, ivermectin, eprinomectin, abamectin, or moxidectin. In other embodiments, the agent may be a nucleic acid.

In a related embodiment, the method may further comprise decreasing the extracellular chloride concentration relative to that in the subject cell to promote efflux of chloride ions from the subject cell, thereby altering the membrane potential of the subject cell. In yet other related embodiments, the method may further comprise increasing the extracellular chloride concentration relative to that in the subject cell to promote influx of chloride ions to the subject cell, thereby altering the membrane potential of the subject cell. In either embodiments, the subject cell may become depolarized or hyperpolarized.

In certain embodiments, the subject cell may be a progenitor cell. In certain embodiments, the progenitor cell may be a neural crest stem cell, e.g., a melanocyte precursor cell. In some embodiments, the progenitor cell may be a human mesenchymal stem cell, neural stem cell, a neuroectodermal stem cell, or an embryonic stem cell.

In other embodiments, the subject cell may be a cancer cell, e.g., melanoma cell.

In some embodiments, the subject cell may be a neural crest derivative, e.g., melanocytes, or melanocyte precursor.

In other embodiments, any of the foregoing methods promote proliferation, differentiation, and/or migration of the subject cell. In certain embodiments, any of the foregoing methods inhibit proliferation, differentiation, and/or migration of the subject cell. In other related embodiments, any of the foregoing methods may inhibit migration of the subject cell.

In any of the foregoing embodiments, the method may comprise administering one or more GlyCl channel modulator to a culture comprising the progenitor cell. In some embodiments, the progenitor cell endogenously expresses the GlyCl channel, or the progenitor cell comprises a transgene expressing the GlyCl channel, or both.

In any of the foregoing embodiments, the method may comprise administering one or more GABA(A) channel modulator to a culture comprising the progenitor cell. In some embodiments, the progenitor cell endogenously expresses the GABA(A) channel, or the progenitor cell comprises a transgene expressing the GABA(A) channel, or both.

In another aspect, the invention provides a method of modulating proliferation, differentiation, and/or migration of a subject cell, comprising administering an effective amount of an agent to alter the membrane potential of the subject cell, thereby modulating the proliferation, differentiation, and/or migration of the subject cell through an endogenous ligand-gated channel.

In certain embodiments, the ligand-gated channel may be a chloride channel, e.g., a glycine-gated chloride (GlyCl) channel, or a GABA(A) channel. In some embodiments, the agent may be a small molecule that modulates the ligand-gated channel. For example, the agent may be a macrocyclic lactone selected from at least one member of the group: avermectin, ivermectin, eprinomectin, abamectin, or moxidectin. In other embodiments, the agent may be a nucleic acid. In certain embodiments, the agent may be muscimol. In other embodiments, the method comprises administering an agent to a culture comprising the subject cell. In any of the foregoing aspects and embodiments, the ligand-gated channel may be a K_{ATP} channel, e.g., Kir6.1- or Kir6.2-type subunits with sulfonylurea receptors (SUR). By way of example, small molecules such as diazoxide, pinacidil, or glibenclamide can be used as modulators of K_{ATP} channels.

In a related embodiment, the method may further comprise decreasing the extracellular ionic concentration relative to that in the subject cell to promote ion flux of the subject cell, thereby altering the membrane potential of the subject cell. In other embodiments, the method may further comprise increasing the extracellular ionic concentration relative to that in the subject cell to promote ion flux of the subject cell, thereby altering the membrane potential of the subject cell. In either embodiments, the subject cell may become depolarized or hyperpolarized.

In certain embodiments, the subject cell is a progenitor cell. In a related embodiment, the progenitor cell may be a neural crest stem cell, e.g., a melanocyte precursor cell. In some embodiments, the progenitor cell may be a human mesenchymal stem cell, neural stem cell, a neuroectodermal stem cell, or an embryonic stem cell.

In some embodiments, the subject cell is a cancer cell, e.g., melanoma cell. In other embodiments, the subject cell is a neural crest derivative, e.g., melanocytes or melanocyte precursor.

In other embodiments, any of the foregoing methods promote proliferation, differentiation, and/or migration of the subject cell. In certain embodiments, any of the foregoing methods inhibit proliferation, differentiation, and/or migration of the subject cell. In other related embodiments, any of the foregoing methods may inhibit migration of the subject cell.

In another aspect, the invention provides a method of promoting tissue regeneration, comprising administering an effective amount of an agent to alter the membrane potential of an instructor cell, which instructor cell endogenously expresses a GlyCl chloride channel, wherein said agent alters the membrane potential of the instructor cell through said endogenously expressed GlyCl channel, thereby promoting tissue regeneration.

In a related embodiment, the method may further comprise decreasing the extracellular chloride concentration relative to that in the instructor cell to promote efflux of chloride ions from the instructor cell, thereby altering the membrane potential of the instructor cell. In other embodiments, the method may further comprise increasing the extracellular chloride concentration relative to that in the instructor cell to promote influx of chloride ions to the instructor cell, thereby altering the membrane potential of the instructor cell. In either embodiment, the instructor cell may become depolarized or hyperpolarized. In some embodiments, the method comprises administering the agent to a culture comprising the instructor cell.

In certain embodiments, the agent is a macrocyclic lactone selected from the group: avermectin, ivermectin, eprinomectin, abamectin, or moxidectin. In other embodiments, the agent is a nucleic acid or a small molecule.

In certain embodiments, any of the foregoing methods promotes proliferation, differentiation, and/or migration of a progenitor cell. In other embodiments, the method inhibits migration of a progenitor cell.

The invention contemplates combinations of any of the foregoing or following aspects and embodiments of the invention.

### BRIEF DESCRIPTION OF THE DRAWINGS

FIG. 1. Ivermectin exposure induces hyper-pigmentation. (A) Control embryos display a medially concentrated pigment pattern with the lateral eyefield being largely devoid of melanocytes. (B) Embryos exposed to the chloride channel inhibitor ivermectin develop a hyper-pigmented phenotype in the presence of otherwise normal development. (C) The ivermectin induced phenotype is highly penetrant, with 98 percent of treated embryos developing hyper-pigmentation. (D) Migration could be blocked by the drug 84093 (obtained from an NIH drug library and discussed in Mol. Biosyst., 2009, 5(4): 376-84. Epub 2009 Feb. 3), although the melanocytes maintained their dendritic morphology.

FIG. 2. Early ivermectin exposure induces an increase in pigment cell proliferation. Embryos exposed to ivermectin from stages 10-24 or 28-46 both show darkening due to expansion of melanocytes. To determine if there was also a corresponding increase in melanoctye number, photographs were taken of controls (A) and ivermectin exposed (B) embryos after tricane anesthetization which contracts the pigment cells. The numbers of melanocytes in the eye field (red boxes) were then counted. Early exposed embryos showed a 1.5× increase in melanocyte number relative to controls (C), whereas no detectible difference was observed between late exposed embryos and controls.

FIG. 3. Glycine exposure induces hyper-pigmentation. Compared to wild type embryos (A, B) exposure to the GluCl/GlyCl receptor ligand glycine throughout development induces hyper-pigmentation at a similar level to ivermectin exposure (C, D)

FIG. 4. Expression of GlyCl subunit alpha mRNA (GenBank accession codes CX801861 and CX796670—GlyCl receptor alpha subunit). In situ hybridization was performed on Xenopus embryos throughout development with an antisense probe to GlyCl channel subunit alpha. (A) Expression was first detected during neurulation in the developing neural plate. (B) Expression continued pan-neurally at stage 17, with the entire neuro-ectoderm showing staining (C, arrows). Expression became restricted during later development (D) with foci of staining observed in the ventral neural tube in the anterior of the animal (E) and the dorsal neural tube in the posterior of the animal (F). In addition, specific punctate expression was observed within the lateral mesoderm of late tailbud embryos (G, arrows).

FIG. 5. Inhibition of ductin induces hyperpigmentation. Microinjection of a dominant negative form of ductin (dn-xDuct) at the one cell stage inhibits a hyperpolarizing H+-V-ATPase and results in hyperpigmentation (A,B). Injections result in hyper-pigmented tadpoles in 11.5 percent of embryos (C), significantly higher then background levels observed in control embryos. Hyper-pigmented embryos arising from dn-xDuct injections were photographed and the number of melanocytes in the eye field counted. There was a 2.1 fold increase in number of melanocytes between hyper-pigmented injected embryos and age matched controls (D).

FIG. 6. Human melanocytes exhibit dendritic morphology under membrane depolarization. In normal culture media, human melanocytes typically develop with two or three projections (A). When grown in media supplemented with 50 mM potassium gluconate, cells develop a more dendritic morphology, with many cells having four, five, or more projections. Comparisons between treatments (C) demonstrate a significant effect of potassium gluconate on dendricity of melanocytes. Image analysis comparison with membrane voltage sensors CC2-DMPE and DiBAC₄ between controls (D) and high potassium cultured cells (E) revealed a depolarization in response to the high potassium media (F)

FIG. 7. Inhibition of voltage sensitive phosphatase (VSP) signaling induces hyper-pigmentation. Compared to controls (A), microinjection of a hairpin RNAi targeting VSP results in hyper-pigmented embryos (B). When quantified, hyper-pigmented embryos showed a significant 2.2 fold increase in melanocyte number compared to not hyper-pigmented siblings (C).

FIG. 8 Inhibition of GlyCl channel causes a phenotypic change in the cement gland, a neuroectodermal derivative. Compared to control embryos (A), strychnine treated Xenopus embryos (B, C, D) exhibited expansion of the cement gland (see arrows indicating cement glands).

### DETAILED DESCRIPTION OF THE INVENTION

### I. Overview

Much of regenerative medicine is focused on stem cells (Chen et al., 2008; Cuenca-Lopez et al., 2008). Most applications currently focus on biochemical factors in the external medium and expression of transgenes to control differentiation and proliferation of stem cells (Dan and Benvenisty, 2006; Ingber and Levin, 2007). Recently, techniques using bioelectrical signals have been developed and applied (Adams and Levin, 2006a, 2006b; Levin, 2007) to modulate stem cell behavior. In particular, the ability to control proliferation, migration, and differentiation in human mesenchymal stem cells and tadpole neural crest derivatives have been shown (Morokuma et al., 2008a; Oviedo and Levin, 2007; Sundelacruz et al., 2008). In a number of cases, bioelectrical control of large-scale patterning phenomena highly relevant to the induction of regeneration of whole organs or appendages has been demonstrated (Adams et al., 2007; Levin et al., 2002; Nogi and Levin, 2005).

The present invention provides, in part, a novel technique for the bioelectrical modulation of cells without requiring expression of heterologous transporter proteins. Instead, ion channels/pumps already expressed in the cells are used. The endogenous channels can be opened or closed using, for example, a small molecule, and its ion flux can be controlled by modulating the extracellular milieu. As exemplified herein, the drug ivermectin (approved for human and veterinary use as an anti-parasitic agent) may be used to open the glycine-gated chloride (GlyCl) channel. Once open, the extracellular chloride concentration can be decreased to depolarize the transmembrane potential of the cells (negative chloride ions leave through this channel, down the concentration gradient). Alternatively, the extracellular chloride concentration can be increased to hyperpolarize the membrane voltage of the cells (negative chloride ions will enter the cell through this channel, down the concentration gradient). Similarly, the extracellular chloride concentration can be appropriately altered to repolarize (i.e., revert back to resting state) the membrane voltage of cells that are abnormally depolarized or hyperpolarized. Thus, by opening an endogenously expressed channel using an agent, e.g., a small molecule, and then modulating the extracellular environment appropriately, the membrane potential of cells can be controlled in vitro or in vivo in a time-dependent manner to control any one or more of proliferation, differentiation, migration, neural outgrowth, and appendage regeneration. Similarly, as shown herein, the endogenous ion channel can be closed using a second small molecule, for example strychnine, to modulate the membrane potential and cell behavior.

The novel techniques described herein can be used to modulate membrane potential and cell behavior by (a) modulating the membrane potential of an instructor cell such that the instructor cell can influence a responder cell (the “subject cell”) and/or (b) modulating the membrane potential of a stem cell (the “subject cell”) which can itself proliferation, differentiate, and/or migrate as a consequence of the change in membrane potential. In some embodiments, membrane potential modulation of the instructor cell and/or the stem cell may be fine-tuned to promote or inhibit proliferation, differentiation and/or migration of a subject cell. For example, as described herein, depolarizing an instructor cell or a stem cell promotes proliferation, differentiation and/or migration of a subject cell. Such an effect may be desirable in tissue generation/regeneration, where proliferation of a tissue type or differentiation of a precursor cell into a desired tissue type is sought. In certain embodiments, however, the constitutively depolarized state of an instructor or stem cell may lead to overproliferation, contributing to a cancer-like state. Accordingly, in such instances, it is desirable to repolarize or restore (i.e., revert back to resting state), or hyperpolarize the depolarized state of an instructor or stem cell to inhibit proliferation.

In a related embodiment, disclosed herein is a novel approach by which cells can be stimulated to proliferate, differentiate, and/or migrate (for example, to generate or regenerate) into a desired tissue type. Specifically, the present invention relates, in part, to the discovery that a population of cells, termed “instructor cells”, play a role in tissue generation and/or regeneration by “instructing” cells non-cell-autonomously. The instructor cells may influence the development of cells directly, or indirectly, e.g., by influencing a signaling cascade. Without being bound by theory, each population of instructor cells is identifiable by endogenous expression of one or more ion channel proteins. Modulation of ion flux and membrane potential of the instructor cell via that channel protein triggers the instructor cell to modulate, directly or indirectly, proliferation, differentiation, and/or migration of other cells, i.e., subject cells (e.g., responder cells). The cells influenced by a given instructor cell population may, in certain embodiments, be located at a distance from the instructor cells. In a related embodiment, membrane potential of progenitor cells can be directly modulated to influence the behavior of the stem cells. In another related embodiment, the present disclosure provides a novel approach by which proliferation, differentiation and/or migration can be inhibited.

In certain aspects, the present invention also provides a method for identifying candidate therapeutic agents that are useful for proliferating, differentiating, and regenerating any tissue type desired.

The present invention has numerous utilities. For example, the present invention provides unique markers (e.g., GlyCl) of a population of instructor cells. Further, the present invention provides methods for promoting proliferation, differentiation, and/or survival of cells, including progenitor cells. Additionally, the present invention provides methods for inhibiting proliferation, differentiation, and/or survival of cells. Such methods may be used in vitro or in vivo to modulate cell behavior and/or promote tissue generation or regeneration. Alternatively, the present methods may be used to inhibit tissue generation/proliferation (i.e., anti-tumor methods), thereby providing a novel mechanism by which bioelectrical properties of the microenvironment mediate the stem cell-cancer cell transition. Further, the present invention provides methods for identifying other populations of instructor cells that can be used to influence the behavior of other cell types.

### II. Definitions

The articles “a” and “an” are used herein to refer to one or to more than one (i.e., to at least one) of the grammatical object of the article. By way of example, “an element” means one element or more than one element.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The term “wild type” refers to the naturally-occurring polynucleotide sequence encoding a protein, or a portion thereof, or protein sequence, or portion thereof, respectively, as it normally exists in vivo. The term “wild type” also refers to a phenotypically and genotypically normal organism.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent). The term “mutant” also refers to an organism with one or more phenotypic or genotypic alterations in comparison to a wild type organism of the same species.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The terms “compound,” “modulator” and “agent” are used interchangeably to refer to nucleic acids, peptides, polypeptides, or small molecules. In the context of the present invention, compounds or agents may modulate ion flux, for example, by inhibiting or promoting ion flux mediated by a particular ion transporter protein or class of ion transporter proteins. Exemplary nucleic acid agents include, but are not limited to, sense or antisense nucleic acids, sense or antisense oligonucleotides, ribozymes, and RNAi constructs. Exemplary peptide and polypeptide agents include growth factors, transcription factors, peptidomimetics, and antibodies, as well as particular ion transporter proteins or subunits thereof. Exemplary small molecules include small organic or inorganic molecules, e.g., with molecular weights less than 7500 amu, preferably less than 5000 amu, and even more preferably less than 2000, 1500, 1000, or 500 amu. One class of small molecules is a class of macrocyclic lactones, which includes, for example, avermectin, ivermectin, eprinomectin, abamectin, or moxidectin.

A “marker” is used to determine the state of a cell. Markers are characteristics, whether morphological or biochemical (enzymatic), particular to a cell type, or molecules expressed by the cell type. A marker may be a protein marker, such as a protein marker possessing an epitope for antibodies or other binding molecules available in the art. A marker may also consist of any molecule found in a cell, including, but not limited to, proteins (peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, and the ability to differentiate along particular lineages.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope.

“Differentiation” describes the acquisition or possession of one or more characteristics or functions different from that of the original cell type. A differentiated cell is one that has a different character or function from the surrounding structures or from the precursor of that cell (even the same cell). The process of differentiation gives rise from a limited set of cells (for example, in vertebrates, the three germ layers of the embryo: ectoderm, mesoderm and endoderm) to cellular diversity, creating all of the many specialized cell types that comprise an individual.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway. In many, but not all tissues, the process of differentiation is coupled with exit from the cell cycle. In these cases, the cells typically lose or greatly restrict their capacity to proliferate and such cells are commonly referred to as being terminally differentiated.

The term regeneration refers to the restoration of cells, tissues, or structures following injury, ablation, loss, or disease. Regeneration involves an interplay of proliferation, differentiation, sometimes dedifferentiation, and migration of cells, alone or in any combination. In some instances, regeneration refers to individual cells or groups of cells. In other instances, regeneration comprises restoration of all or a portion of a tissue or organ. The invention provides methods of promoting or enhancing regeneration. In some embodiments, the method of promoting or enhancing regeneration includes modulating one or more of proliferation, differentiation, dedifferentiation, survival, or migration.

As used herein, the term “population of cells” refers to one or more cells in a tissue, organ, or culture. A population of cells may be manipulated or examined in vivo or in vitro.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues.

The term “embryonic stem cell” is used to refer to the pluripotent stem cells of, for example, the inner cell mass of the mammalian embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

The term “adult stem cell” is used to refer to any multipotent stem cell derived from tissues other than the embryonic blastocyst. Adult stem cells include cells derived from non-blastocyst tissue, including tadpole, fetal, juvenile, and adult tissue. Stem cells have been isolated from a wide variety of adult tissues (e.g., non-blastocyst) including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the invention contemplates the identification of progenitor cells resident in any tissue, in any organism, during any stage of development.

The term “instructor cell” is used to refer to a population of cells capable of directing the proliferation, differentiation, and/or migration of another population of cells responsive to the instructor cell (responder cells). As described herein, altering the membrane potential of instructor cells can control the ultimate fate of cells on which the instructor cells act directly or indirectly. The nature of instructor cells can vary widely, and may include, for example, stem cells or partially or terminally differentiated cells. Moreover the cells upon which a given population of instructor cells act may be progenitor cells, partially differentiated cells, or terminally differentiated cells. Such cells may be located at some distance from the instructor cells. A population of instructor cells is identifiable based on endogenous ion channel expression. Without being bound by theory, it is expected that each instructor cell modulates the cellular behavior of a relatively narrow range of other cell types.

As used herein, the term “subject cell” is used to refer to a cell which is ultimately influenced—the subject of the membrane potential modulation. That is, as described herein, for a scenario in which an instructor cell's Vₘₑₘ is modulated to produce an effect on a responder cell, the subject cell is the responder cell. However, in certain instances where a cell's Vₘₑₘ is directly modulated to produce a phenotypic effect, the modulated cell is the subject cell. For example, as described herein, when the membrane potential of a progenitor cell is modulated to produce a phenotypic effect, the progenitor cell is the subject cell.

As used herein, the term “effective amount” means the total amount of the active component(s) of a composition or compound that is sufficient to cause a statistically significant change on a detectable biochemical or phenotypic characteristic. When applied to an individual active ingredient, administered alone, the term refers to that ingredient alone. When applied to a combination, the term refers to combined amounts of the active ingredients that result in the effect, whether administered in combination, serially or simultaneously.

The term “membrane” refers to phospholipid bilayers, which includes but is not limited to the cell membrane as well as other membranes of intracellular organelles, e.g., nucleus, golgi, mitochondria, etc.

The term “ion flux” refers to the movement of ions through an area/unit time. The term does not imply anything about the mechanism of ion movement. The term includes ion flux mediated by any ion transporter protein regardless of whether the transporter protein actively or passively shuttles ions. The term ion flux includes movement of ions into a cell or movement of ions out of a cells (e.g., efflux or influx). As used herein, the terms “ion transporter proteins,” “transporter proteins,” “protein pumps” and “channel proteins” are used interchangeably and include proteins that mediate ion flux regardless of the particular ion species transported or the particular mechanism of action. The term includes proteins that are passive transporters, as well as proteins that are active transporters. “Class of ion transporter proteins” refers to categories of transporter proteins organized based on similar functional characteristics. For examples, a class of ion transporter proteins may include transporter proteins that transport a particular ion species (e.g., Ca, Cl, Na, H) or transporter proteins that transport a particular ion species using a particular mechanism of action.

### III. Detailed Description of Illustrative Embodiments

One of the goals of studying regeneration has been to understand the principles and processes that modulate regeneration in species capable of mounting robust regenerative responses, so that these principles and processes can be applied to increase the regenerative capacity of other organisms. The present disclosure provides the following novel concepts and methods by which tissue generation and regeneration may be achieved. In a similar vein, understanding the principles and processes that enhance regeneration (i.e., cellular proliferation) provides, in turn, methods by which cellular proliferation may be reversed or inhibited and, further, differentiation or migration. That is, the present methods may be similarly applied in anti-cancer therapies by inhibiting cellular proliferation. Moreover, the present disclosure provides methods by which migration of metastatic cancer cells may be inhibited. Accordingly, the present methods are applicable to both regenerative and anti-proliferative therapeutics.

### A. Methods of Identifying Instructor Cells

This aspect of the present invention is based, in part, on our findings indicating that a population of cells, termed “instructor cells”, can direct the proliferation, differentiation, and/or migration, and ultimately the fate, of neighboring and/or distant cells. Without being bound by theory, the cells upon which a given instructor cell acts may be progenitor cells or partially or terminally differentiated cells. As exemplified herein (Example 1), we have identified an example of an instructor cell population capable of influencing melanocyte (a neural crest derivative), as well as neuroectodermal fate. This discrete instructor cell population expresses the glycine-gated chloride (GlyCl) channel. Briefly, one arm of the results showed that when Xenopus embryos from gastrulation throughout development were treated with ivermectin, a compound known to activate the GlyCl channel, 98% of the treated larvae developed a striking hyperpigmentation. Further, the melanocytes in treated individuals exhibited a dendritic morphology and often migrated to regions normally devoid of pigment cells, such as the lateral eye field and base of the tail. In the second arm of the study (Example 1B), the embryonic source of the hyperpigmentation signal was sought. That is, the expression pattern of GlyCl was observed throughout various stages of embryonic development. This revealed that, since numerous cells in the embryo express the target receptor, the effect of ivermectin is unlikely to be only directly on the melanocytes themselves, and supports the conclusion that ivermectin likely acts on a population of cells described herein as instructor cells. Without being bound by theory, the instructor cells non-cell-autonomously induce certain cells to differentiate and/or migrate, and otherwise determine the fate of a cell in the process of generating melanocytes.

Accordingly, one aspect of the invention provides a method of identifying a population of instructor cells that modulate (i.e., promote or inhibit) proliferation, differentiation, and/or migration of a cell, comprising contacting a sample with an agent that detects expression of a GlyCl channel, wherein cells in the sample that express the GlyCl channel are identified as instructor cells that modulate proliferation, differentiation, and/or migration of a cell, such as a melanocyte, a melanocyte precursor, a neural crest derivative cell, a neural precursor, a neural derivative cell, or a neuroectodermal cell. An instructor cell may be a particular population of stem cells, or a population of cells more differentiated than a stem cell. For example, an instructor cell may be a neural crest derivative, mesenchymal stem cell, or a melanocyte. Similarly, a cell that is acted upon by an instructor cell, either directly or indirectly, may also be any number of stem cells, or a population of cells more differentiated than a stem cell, as defined herein.

It should also be understood that, as described herein, channel proteins other than GlyCl may serve as markers for other instructor cells. For example, other markers include channel proteins that transport the following ion species: K, Na, Ca, H, Cl, Zn, Cu, Fe, OH, HCO₃. Different instructor cell populations likely function to influence the proliferation, differentiation, and/or migration of distinct responder cell populations. Thus, identifying a range of instructor cell populations provides a mechanism for modulating behavior of a range of responder cell populations.

The experiments described herein summarize the identification of a population of instructor cells that expressed a GlyCl channel. This population of instructor cells acts with great specificity to influence proliferation, differentiation, and migration of melanocytes and/or melanocyte precursors. Without being bound by theory, it is contemplated that other instructor cell populations capable of modulating cell behavior of other cell types (e.g., cell types that contribute to other tissues) will be characterized and identifiable based on a unique pattern of endogenous expression of one or more ion channels.

In a related embodiment, an agent that detects a marker channel may be one that detects the marker protein or marker mRNA. Some examples of such agents include, for example, antibodies or nucleic acid probes that bind to the marker protein or hybridize to a nucleic acid encoding the marker protein, respectively. For example, and as exemplified herein, antibodies or nucleic acid probes that detect, specifically, the GlyCl channel may be used. Reagents and methods for detection of protein or mRNA in a sample are well known in the art.

In certain aspects of the invention, also provided herein is a method for identifying an instructor cell capable of modulating proliferation, differentiation, and/or migration of a progenitor cell, comprising: i) identifying one or more ligand-gated channels endogenously expressed by a discrete population of candidate instructor cells, ii) administering an effective amount of an agent to alter membrane potential of the candidate instructor cell through the identified ligand-gated channel, and iii) assaying for the presence or absence of a desired phenotype, whereby the presence or absence of a desired phenotype indicates that the candidate instructor cell is an instructor cell capable of modulating proliferation, differentiation, and/or migration of one or more responder cells.

In this approach, the user may screen for the expression of a channel protein of choice from a panel of channel proteins known in the art (e.g., GlyCl, H+-V-ATPase, K-ATP channel, Na/Ca exchanger, etc., known in the art) using, e.g., in situ hybridization, or any method known in the art. By doing so, one or more population of candidate instructor cells that each constitute a discrete pattern of expression for a given channel protein may then be selected for further analysis. Since the identity of the endogenous channel protein for each candidate instructor cell is known, the appropriate agent that modulates (opens or closes) the particular channel protein can be administered to screen for any number of desired phenotypic traits. By way of example, as demonstrated in Example 1B of the present invention, hyperpigmentation and proliferation of melanocytes (a neural crest derivative) in an embryo may be assessed.

Accordingly, the present method allows for the identification of candidate therapeutic agents that may be used to promote or inhibit proliferation, differentiation, and regeneration of any number of tissue type desired. Further, and as reflected in the Examples and described herein, the identified instructor cells influence responder cells with a high level of specificity. For example, modulating membrane potential via an endogenously expressed GlyCl channel resulted in a pronounced effect on melanocytes, without observable effects on other neural crest derivatives. Thus, the approach and methods provided herein allows manipulation of cells and tissues with a high level of specificity by harnessing endogenous channel expression and instructor cell activity. These principles of instructor cells are applicable to other aspects of modulating membrane potential of instructor cells described throughout the application.

B. Methods of Modulating Proliferation, Differentiation, and/or Migration

### (i) Altering the Membrane Potential of Instructor Cells to Control Cell Behavior

In certain aspects, the disclosure provides a method of promoting tissue regeneration by administering an effective amount of an agent to alter the membrane potential of an instructor cell, wherein the agent alters the membrane potential of the instructor cell through an endogenous ligand-gated channel expressed in the instructor cell, thereby promoting tissue regeneration. Tissue regeneration may be the result of proliferation, differentiation, and migration, alone or in any combination thereof, of a cell on which the instructor cell acts non-cell-autonomously. That is, by altering the membrane potential of a population of instructor cells, the fate of the cells upon which the instructor cells act directly or indirectly (responder cells) can be controlled as desired. The methods of the present invention allow the use of channel proteins endogenously expressed in the instructor cells, without the need to express or misexpress any exogenous channel protein genes. The instructor cells may be located at some distance from the cells upon which they act (e.g., the instructor cell may not be in cell-to-cell contact with the cells upon which they exert their effects).

Without being bound by theory, instructor cells provide a small pool of cells that can be used to influence cell behavior without necessarily altering the cell fate of the instructor cell itself. Thus, one characteristic of this aspect of the invention is that modulation of membrane potential in one cell type (e.g., the instructor cell) is used to influence the phenotype and growth characteristics of another distinct cell type (e.g., the cells acted upon by the instructor cells).

The cell types acted upon by the instructor cells may be progenitor cells, or partially or terminally differentiated cell types. For example, the cell type may be an embryonic stem cell or adult stem cell. By way of further example, the cell type acted upon may be a neural crest derivative, a neural derivative, a melanocyte precursor, a neuroectodermal derivative.

The instructor cell may also be a progenitor cell, or a partially or fully differentiated cell.

Additionally, the invention contemplates the use of the present methods to promote proliferation, differentiation, and/or migration of epithelial tissue, muscle tissue, nervous tissue, or connective tissue. That is, the present methods may be applied to generate epithelial tissue, muscle tissue, nervous tissue, or connective tissue as described herein, by modulating instructor cells according to the invention which ultimately “instruct” other cells (responder cells) to proliferate and/or differentiate into the desired tissue type. For example, by modulating a population of instructor cells according to the invention, melanocytes or melanocyte precursors can be induced to proliferate and differentiate into more melanocytes, or even into axonal cells.

In another related aspect of the invention, provided herein is a method for modulating proliferation, differentiation, and/or migration of one or more cells, comprising: i) identifying an instructor cell capable of modulating proliferation, differentiation, and/or migration of the cell, ii) identifying one or more ligand-gated channels endogenously expressed in the instructor cell, and iii) administering an effective amount of an agent to alter membrane potential of the instructor cell through the ligand-gated channel, whereby the instructor cell modulates proliferation, differentiation, and/or migration of one or more cells. The cells whose behavior is influenced by the instructor cells are referred to as responder cells.

As outlined in the previous section, in one embodiment, a population of instructor cells may be identified by determining the population of cells in a sample that endogenously express the GlyCl channel. Alternatively, an instructor cell may also be identified by determining the population of cells in a sample that endogenously express a channel protein other than GlyCl, such as, e.g., a potassium channel or another chloride channel. Upon identification of a population of instructor cells, other ligand-gated channels endogenously expressed in the instructor cell may also be identified using the methods described herein. Once one or more channels are identified, an effective amount of appropriate agents, e.g., small molecules or nucleic acid constructs as described herein, that alter channel activity may be administered to modulate the membrane potential of instructor cells as desired, thereby modulating proliferation, differentiation, and/or migration of one or more cells.

In a related embodiment, any of the foregoing methods may further comprise modulating the extracellular ionic concentration relative to that in the instructor cell to promote influx or efflux of ions to and from the instructor cell, respectively, thereby altering the membrane potential of the instructor cell. For example, if one desires to alter the chloride ion concentration through a chloride channel, e.g., GlyCl channel, then the extracellular chloride concentration may be decreased to promote efflux of chloride ions from the instructor cells, thereby depolarizing the instructor cell. Alternatively, in this particular example, the extracellular chloride concentration may be increased to promote influx of chloride ions from the instructor cells, thereby hyperpolarizing the instructor cell. Depending on the nature of the instructor cell as well as the nature of the membrane polarization, various phenotypic changes may be effected.

As exemplified herein, depolarizing the membrane potential of a particular population of instructor cells through the GlyCl channel induces proliferation, differentiation, and migration of melanocytes (a neural crest derivative), as evidenced by the hyperpigmented phenotype of Xenopus embryos shown in Example 1. Uncontrolled growth and dispersal of melanocytes can lead to melanoma. Melanoma cell migration, as well as migration of other cell types, is known to be dependent on K⁺ channels, and a number of ion channels have been characterized as markers and likely causes of neoplasm. In fact, some ion channels (e.g., the oncofetal form of NaV1.5 sodium channel) are oncogenes. The present disclosure suggests that control of membrane voltage is a key component of this set of pathways, and may be a promising target for melanoma treatment, as well as other types of neoplasm. Furthermore, membrane voltage may be an important and novel regulator of the stem cell-cancer cell transition (i.e., stem cells that progress into cancer cells), and the present disclosure provides support for this concept. That is, and as further detailed herein, the present disclosure demonstrates the use of ivermectin to depolarize a population of cells (the instructor cells) in the environment of stem cells, in particular a neural crest derivative, to cause the stem cells to become cancerous.

As such, it may be possible for early, non-invasive cancer detection using, e.g., voltage-sensitive fluorescent reporter dyes and techniques to normalize cancer by repolarizing or hyperpolarizing neoplastic cells and instructor cell populations, as described herein. For example, the membrane potential of a population of instructor cells may be measured non-invasively, and further repolarized or hyperpolarized according to the present methods to inhibit proliferation (i.e., prevent or treat cancer). In a related embodiment, a cancerous cell may be non-invasively identified by measuring its membrane potential (i.e., a depolarized state). The identified cells (i.e., the population of instructor cells or the cancerous cells), may be repolarized or hyperpolarized to normalize, treat, or prevent cancer. In a specific embodiment, the present disclosure may be applied in the detection, prevention, and/or treatment of melanoma. In a further related embodiment, and as detailed herein, instructor cells and/or subject cells may be depolarized according to the present methods to increase tissue proliferation and stem cell growth for regenerative applications.

For any of the foregoing methods, it is understood that the present methods may be applied to any number of channel proteins known in the art. For example, agents that modulate channel proteins involved in the transport of other ions, e.g., K, Na, Ca, H, Cl, Zn, Cu, Fe, OH, HCO₃, expressed in instructor cells may be used as appropriate.

In some embodiments, it is understood that the instructor cell need not be in direct contact with the cell or tissue for which proliferation, differentiation, and/or migration is promoted or inhibited (e.g., the instructor cell need not be in direct contact with the cell upon which the instructor cell acts directly or indirectly to promote proliferation, differentiation, and/or survival). In certain embodiments, promoting proliferation, differentiation, and/or survival promotes tissue generation or regeneration.

### (ii) Altering the Membrane Potential of Subject Cells to Control Cell Behavior

In certain aspects, the invention provides methods of promoting or inhibiting proliferation, differentiation, migration, and/or survival of a subject cell. In contrast to methods described above for modulating membrane potential of an instructor cell to influence behavior of a separate cell type (i.e., the responder cell), in this aspect, membrane potential is modulated to influence behavior of that same cell, or the “subject cell” (e.g., influence the same cell whose membrane potential is being modulated). That is, the cell whose membrane potential is modulated is also the cell exhibiting the desired effect.

In certain embodiments, the subject cell endogenously expresses a particular ion channel, and that channel is used to modulate membrane potential of the progenitor cell. In other embodiments, expression of a particular ion channel is increased by exogenously expressing an ion channel in the subject cell. In other embodiments, the subject cells are grown in co-culture (either in contact or without contact) with cells that express the ion channel of interest, and those cells serve as instructor cells for the subject cells.

In some embodiments, the subject cell is a progenitor cell for which membrane potential is modulated according to the present methods to promote or inhibit proliferation, differentiation, migration, and/or survival. In other embodiments, the subject cell is a cancerous cell for which membrane potential is modulated according to the present methods to inhibit proliferation, differentiation, migration, and/or survival.

(iii) Modulation of GlyCl Channel Expressed in Progenitor Cell to Control the Fate of Progenitor Cells

In certain aspects, the present invention provides a method of modulating (i.e., promoting or inhibiting) proliferation, differentiation, and/or migration of a progenitor cell, wherein the progenitor cell expresses a GlyCl channel, comprising administering to the progenitor cell an effective amount of a GlyCl channel modulator to alter the membrane potential of the progenitor cell, thereby modulating the proliferation, differentiation, and/or migration of the progenitor cell. Similar to the methods described for instructor cells in the preceding sections, the present method may be applied directly to progenitor cells that express the GlyCl channel. That is, using the GlyCl channel expressed in the progenitor cells, the membrane potential of these cells can be directly altered using, e.g., ivermectin, to modulate the proliferation, differentiation, and/or migration of progenitor cells.

As before, the method may further comprise modulating the extracellular ionic concentration relative to that in the progenitor cell to promote influx or efflux of ions to and from the progenitor cell, respectively, thereby altering the membrane potential of the progenitor cell. For example, if one desires to alter the chloride ion concentration through a chloride channel, e.g., GlyCl channel, then the extracellular chloride concentration may be decreased to promote efflux of chloride ions from the progenitor cells, thereby depolarizing the progenitor cell. Alternatively, in this particular example, the extracellular chloride concentration may be increased to promote influx of chloride ions from the progenitor cells, thereby hyperpolarizing the progenitor cell. Depending on the nature of the progenitor cell as well as the nature of the membrane polarization, various phenotypic changes may be effected. It is understood that the method may be applied to any number of channel proteins known in the art.

In some embodiments, the progenitor cell may be a type of stem cell as defined herein, e.g., embryonic stem cell or adult stem cell, or more specifically, neural crest stem cell, or a mesenchymal stem cell.

Additionally, the invention contemplates the use of the present method to promote proliferation, differentiation, and/or migration of epithelial tissue, muscle tissue, nervous tissue, or connective tissue. That is, the present method may be applied to generate epithelial tissue, muscle tissue, nervous tissue, or connective tissue from any progenitor cell as described herein, by modulating the progenitor cells according to the invention to proliferate and/or differentiate into the desired tissue type. For example, by modulating a population of the progenitor cells according to the invention, stem cells may be induced to proliferate and differentiate into, for example, axonal cells.

In certain embodiments, the progenitor cells may express the GlyCl channels endogenously. Alternatively, the progenitor cells may express an exogenous source of (i.e., recombinant) GlyCl channel introduced into the progenitor cells by any of the known recombinant methods known in the art.

Additionally, the present method may be combined with any of the foregoing methods to modulate proliferation, differentiation, and/or migration of progenitor cells. For example, ivermectin may be administered in combination with an agent that alters the membrane potential of an instructor cell (as described in foregoing sections), as appropriate, to promote proliferation, differentiation, and/or migration of progenitor cells.

### (iv) Modulation of One or More Endogenous Channels Expressed in Progenitor Cell to Control the Fate of Progenitor Cells

In certain aspects, the present invention also provides a method of modulating proliferation, differentiation, and/or migration of a progenitor cell, comprising administering an effective amount of an agent to alter the membrane potential of the progenitor cell through a ligand-gated channel endogenously expressed in the progenitor cell, thereby modulating the proliferation, differentiation, and/or migration of the progenitor cell. Similar to the methods described for instructor cells in the preceding sections, the present method may be applied directly to progenitor cells through any ligand-gated channel that is naturally expressed in the progenitor cell. That is, using a preferred ligand-gated channel endogenously expressed in the progenitor cells, the membrane potential of these cells can be directly altered using a channel modulator specific to the endogenously expressed ligand-gated channel to modulate the proliferation, differentiation, and/or migration of progenitor cells.

As before, the method may further comprise modulating the extracellular ionic concentration relative to that in the progenitor cell to promote influx or efflux of ions to and from the progenitor cell, respectively, thereby altering the membrane potential of the progenitor cell. For example, if one desires to alter the chloride ion concentration through a chloride channel, e.g., GlyCl channel, then the extracellular chloride concentration may be decreased to promote efflux of chloride ions from the progenitor cells, thereby depolarizing the progenitor cell. Alternatively, in this particular example, the extracellular chloride concentration may be increased to promote influx of chloride ions from the progenitor cells, thereby hyperpolarizing the progenitor cell. Depending on the nature of the progenitor cell as well as the nature of the membrane polarization, various phenotypic changes may be effected. It is understood that the method may be applied to any number of channel proteins, e.g., H+-V-ATPase, K-ATP channel, Na/Ca exchanger, etc., known in the art.

In some embodiments, the progenitor cell may be a type of stem cell as defined herein, e.g., embryonic stem cell or adult stem cell, or more specifically, neural crest stem cell, or a mesenchymal stem cell.

Additionally, the invention contemplates the use of the present method to promote proliferation, differentiation, and/or migration of epithelial tissue, muscle tissue, nervous tissue, or connective tissue. That is, the present method may be applied to generate epithelial tissue, muscle tissue, nervous tissue, or connective tissue from any progenitor cell as described herein, by modulating the progenitor cells according to the invention to proliferate and/or differentiate into the desired tissue type. For example, by modulating a population of the progenitor cells according to the invention, stem cells may be induced to proliferate and differentiate into, for example, axonal cells.

Additionally, the present method may be combined with any of the foregoing methods to modulate proliferation, differentiation, and/or migration of progenitor cells. For example, an agent that modulates the endogenously expressed channel protein of the present progenitor cells may be administered in combination with an agent that alters the membrane potential of an instructor cell (as described in foregoing sections) to promote proliferation, differentiation, and/or migration of progenitor cells.

Furthermore, the present methods are not limited to the function of the GlyCl protein, nor is it inherently tied to chloride flux. While GlyCl is a convenient target for voltage modulation, the pathway described herein is not limited to any one gene product; rather, it is driven by changes in a biophysical parameter, Vₘₑₘ, that is determined by the activity of multiple transporters. The present disclosure provides a novel control mechanism operating during normal embryogenesis that can regulate neural crest stem cell dynamics, as well as identifying a new environmental parameter that may be involved in neoplastic processes.

TABLE 1
provides exemplary GenBank Accession Numbers
for the alpha subunit of the GlyCl channel.

| Channel/Receptor | GenBank Accession No. |
|---|---|
| GlyCl subunit alpha | CX801861 and CX796670 |
| (Xenopus tropicalis) | |

### IV. Compounds that Modulate Channel Activity

For any of the foregoing methods, the invention contemplates the use of any of a wide range of compounds to open or close a particular channel protein of interest to alter the membrane potential of instructor cells or progenitor cells. Exemplary classes of compounds include, but are not limited to, small organic molecules, small inorganic molecules, nucleic acids (e.g., antisense oligonucleotides, RNAi constructs, ribozymes), peptides, polypeptides, peptidomimetics, and antibodies. Once the channel expressed in the instructor cell or progenitor cell population of interest is identified or known, an appropriate compound can be selected from amongst known modulators of that channel. Alternatively, an appropriate compound can be generated, for example based on the sequence of the nucleic acid that encodes that channel as detailed below.

For example, small compounds or antibodies may bind specifically to a particular channel protein, thereby inactivating ion transport through that channel. The use of several channel modulators, including ivermectin, is exemplified herein. Alternatively, antisense or RNAi constructs may also be used to inactivate a channel protein of interest. Further, certain channels may be preferentially disrupted by expressing a dominant negative form of that particular channel (e.g., YCHE78, a dominant-negative H+-V-ATPase subunit E, as exemplified herein). A few exemplary classes of compounds are described in detail below. Regardless of the specific agent used, in certain embodiments, the agent is used to alter membrane potential by modulating ion flux through an endogenously expressed ion channel.

### A. Small Molecules

Compounds that modulate the activity of particular ion transporters or classes of ion transporters are disclosed in US Application No. 2008/0131920, incorporated herein by reference. Such compounds may be used to modulate various ion channels according to the methods of the present invention. Additionally, disclosed herein is a specific class of molecules known to modulate the GlyCl chloride channel in vertebrates (GluCl in invertebrates): macrocyclic lactones. Non-limiting examples of macrocyclic lactones include avermectin, ivermectin, eprinomectin, abamectin, or moxidectin.

### B. Nucleic Acids

### (i) Antisense, Ribozyme and Triplex Techniques

Nucleic acid-based compounds include, but are not limited to, antisense oligonucleotides and ribozymes. Antisense oligonucleotides and ribozymes inhibit the expression of a protein, e.g., by inhibiting transcription and/or translation.

Binding of the oligonucleotide or ribozyme to the nucleic acid encoding the particular channel protein for which inactivation is desired may be by conventional base pair complementarity, or, for example, in the case of binding to DNA duplexes, through specific interactions in the major groove of the double helix. In general, “antisense” therapy refers to the range of techniques generally employed in the art, and includes any therapy that relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a particular protein. Alternatively, the antisense construct is an oligonucleotide probe that is generated ex vivo and which, when introduced into the cell causes inhibition of expression by hybridizing with the mRNA and/or genomic sequences encoding a particular channel protein. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775).

The antisense oligonucleotide may comprise at least one modified base moiety known in the art. The antisense oligonucleotide may also comprise at least one modified sugar moiety selected from the group including, but not limited to, arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. In yet a further embodiment, the antisense oligonucleotide is an-anomeric oligonucleotide. An-anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides to be used in the present invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451).

The antisense molecules can be delivered to cells or animals in vitro or in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically. These and other methods are have been used to deliver single antisense oligonucleotides, as well as libraries of oligonucleotides.

Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246), and is well known in the art. As in the antisense approach, the ribozymes can be composed of modified oligonucleotides (e.g., for improved stability, targeting, etc.) and can be delivered in vivo or in vitro. Alternatively, endogenous gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Antisense RNA and DNA, ribozyme, and triple helix molecules to be used in the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′O-methyl rather than phosphodiesterase linkages within the oligodeoxyribonucleotide backbone.

### (it) RNAi

In other embodiments, the compound is an RNAi construct. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties, as specifically exemplified herein.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

Production of RNAi constructs to be used in the present invention can be carried out by chemical synthetic methods or by recombinant nucleic acid techniques known in the art. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The siRNA molecules to be used in the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

Exemplary RNAi constructs that specifically recognize a particular gene, or a particular family of genes can be selected using methodology outlined in detail above with respect to the selection of antisense oligonucleotide. Similarly, methods of delivering RNAi constructs include the methods for delivering antisense oligonucleotides outlined in detail above, and as exemplified herein.

### V. Cells and Animals

As outlined throughout in reference to particular methods of the invention, the subject methods can be conducted in vivo or in vitro in cells derived from or resident in virtually any organism.

The foregoing methods can be conducted in cells in culture, in tissue samples maintained ex vivo, or in animals. When the method is conducted using cells in culture, the invention contemplates using cells derived from any organism, tissue, or stage of development. Furthermore, the invention contemplates that the cells may be primary cultures of cells, or transformed cell lines, and that the cells can either be wild type cells or cells containing one or more mutations. Mutant cells or cell lines may be models of a particular disease or injury, or may be derived from animals having a specific disease or injury (e.g. cancer cells harvested from an animal).

Cells may be derived from (e.g., derived from and cultured in vitro as populations of cells or tissues) or reside in (cells resident in a whole animal or portion of a whole animal) any of a number of animal species. Exemplary animals include, but are not limited to, flatworms, amphibians, fish, reptiles, birds, or mammals. Suitable flatworms include planarian. Suitable amphibians include Xenopus laevis, Xenopus tropicalis, and other species of frog. Suitable birds include chickens, as well as other birds commonly used or maintained in a laboratory setting. Suitable mammals include mice, rats, hamsters, goats, sheep, pigs, cows, dogs, cats, rabbits, non-human primates, and humans.

Regardless of the species of cells or animal selected, the invention contemplates that cells may be derived from or reside in an animal of virtually any stage of development. For example, the cells may be derived from or reside in an embryonic, larval, fetal, juvenile, or adult organism.

To further illustrate, in one embodiment, the foregoing methods may be conducted using cells derived from or resident in a nematode. There are over 10,000 known nematode species. These include parasitic nematodes (e.g., nematodes that are parasitic to humans, non-human animals, or plants). Exemplary parasitic nematodes include, but are not limited to, whipworms, Ascaris, hookworms, filarial worms, and root knot nematodes. C. elegans is perhaps the most well known and thoroughly studied nematode, and the invention contemplates using C. elegans or other nematodes.

In another embodiment, the foregoing methods may be conducted in cells derived from or resident in a fish or amphibian species. Zebrafish (e.g., adult zebrafish and developing, e.g., embryonic fish) are a particular example of a fish well suited for study. Zebrafish are an extensively used developmental system, and genetic, cell biological, and molecular biological reagents and methods are well known and available. Additionally, numerous chemical and radiation-based screens have produced large numbers of mutant zebrafish that can also be used for study.

Xenopus laevis and Xenopus tropicalis (e.g., adult, embryonic, tadpole, etc. stage animals) are particular examples of amphibians well suited for study. Both species are used extensively, and well developed reagents exist. For example, the availability of these molecular reagents facilitates assays based on changes in gene or protein expression, either instead of or in addition to assays based on morphological criteria. Additionally, Xenopus tropicalis is a genetically tractable model organism, and mutants have been and continue to be generated and characterized. Xenopus cells and whole organisms are excellent systems for screening assays. The cells of early Xenopus embryos are relatively large, and thus easily manipulated, injected, and used for electrophysiological recording. Eggs and embryos can be collected in very large numbers. This facilitates biochemical, pharmacological, and statistical analyses.

In another embodiment, the foregoing methods may be conducted in cells derived from or resident in a flatworm. Exemplary flatworms are the free-living (e.g., non-parasitic) flatworm planaria. Planaria are in the phylum Platylhelmenthes and the class Turbellaria. There are numerous species of planaria, any of which can be readily used. Planaria exhibit much of the complexity of vertebrate systems: a well-differentiated nervous system, intestine, eyes, brain, three tissue layers, and bilateral symmetry. Planaria represent a critical breakthrough in the evolution of the animal body plan and are thought to very closely resemble the proto-bilaterian ancestor. It is the first organism to have both bilateral symmetry and encephalization, making it capable of detecting environmental stimuli quicker and more efficiently than the lower metazoans. Despite a simplistic appearance and evolutionary position, planaria possess a well-developed nervous system with true synaptic transmission and have what can be considered the first animal “brain” (Sarnat and Netsky (1985) Can J Neurol Sci. 12(4): 296-302). They have also developed sensory capabilities for the detection of light (Brown and Park (1975) Int J. Chronobiol. 3(1):57-62; Brown et al., 1968), chemical gradients (Mason (1975) Anim Behay. May; 23(2): 460-9; Miyamoto and Shimozawa (1985) Zoological Science (Tokyo) 2: 389-396), vibration (Fulgheri and Messeri (1973) Boll Soc Ital Biol Sper. 49(20): 1141-5), electric fields (Brown and Ogden (1968) J Gen Physiol. 51(2)255-60), magnetic fields (Brown and Chow (1975) Physiological Zoology 48: 168-176; Brown (1966) Nature 209: 533-5), and weak y-radiation (Brown and Park (1964) Nature 202: 469-471).

Planaria have exceptional regenerative capacity. A bisected flatworm readily regenerates. Thus planaria, either whole animals or fragments, serve as an excellent model system in which to study the implications of ion flux on cell proliferation, differentiation, and migration, as well as on instructor cell identification and characterization. In addition to planaria, other model systems have enhanced regenerative capacity, and these systems are especially well suited for studies of ion flux on regeneration. By way of example, fish and amphibian species may be especially useful as model systems in such studies.

The invention contemplates the use of animals, including any of the foregoing animals and cells derived therefrom.

Regardless of the particular organism selected, and regardless of whether the subject methods are conducted in vitro or in vivo, cells or animals may be of any developmental stage including, but not limited to, embryonic, fetal, larval, tadpole, juvenile, and adult stage cells or organisms. One of skill in the art can select the proper animal and developmental stage depending on the application of the subject method. Furthermore, one of skill in the art can select the appropriate animal and developmental stage based on the research interests of the investigator, time, and cost considerations, as well as the availability of other complementary research reagents. Additionally, even when whole organisms or large fragments of whole organisms are used, one of skill in the art may choose to examine a particular biological process in only a portion of the whole organism or fragment.

In one embodiment, the animal or tissue (including whole animals, injured animal, fragments, or cell derived therefrom) is selected based on its robust regenerative ability. Cells, tissues, or animals with an enhanced regenerative ability may be useful in methods for identifying and characterizing a role for ion transporter proteins, ion flux, membrane potential, and/or pH in dedifferentiation and regeneration. Exemplary animals and systems with enhanced regenerative capacity include, but are not limited to, planaria, the zebrafish tail, the amphibian (e.g., Xenopus) tail, and the amphibian limb. An understanding of how regeneration is modulated in any of these systems can be used to increase/stimulate regenerative capacity in organisms and systems whose endogenous regenerative capacity is less robust. In another embodiment, the animal or tissue is selected for screening and study specifically because its endogenous regenerative capacity is not robust. Such systems include any cells or tissues derived from organisms, such as mammals, whose endogenous regenerative capacity is not robust. Such systems also include endogenously non-regenerating cells or tissues derived from particular regions of otherwise robustly regenerative organisms.

In one embodiment, the animal/organism is a protochordate. Protochordates possess a hollow dorsal nerve cord, gill slits, and a notochord. Exemplary protochordates include tunicata (e.g., sea squirts, etc.) and cephalochordate (e.g., amphioxus). Exemplary amphioxus include, but are not limited to Ciona intestinalis and Branchiostoma floridae (Holland and Gibson-Brown (2003) BioEssays 25: 528-532; Gostling and Shimeld (2003) Evolution and Development 5: 136; Dehal et al. (2002) Science 298: 2157-2167; Nishiyama et al. (1972) Tohoku J Exp Med 107: 95-96; Ogasawara et al. (2002) Develop Genes Evol 212: 173-185; Pope and Rowley (2002) J Exp Biology 205: 1577-1583).

In another embodiment, the animal/organism is a hemichordate. Exemplary hemichordates include acorn worms (Tagawa et al. (2001) Evol and Develop 3: 443).

In another embodiment, the animal/organism is a nematode. There are over 10,000 known nematode species. These include parasitic nematodes (e.g., nematodes that are parasitic to humans, non-human animals, or plants). Exemplary parasitic nematodes include, but are not limited to, whipworms, Ascaris, hookworms, filarial worms, and root knot nematodes.

C. elegans is perhaps the most well known and thoroughly studied nematode, and the invention contemplates using C. elegans or other nematodes. Although C. elegans is considered a soil nematode, methods for culturing C. elegans in various quantities of liquid media (e.g., in a fluid) are well developed. See, http://elegans.swmed.edu/. Accordingly, the methods and apparatuses of the invention for conducting assays in aquatic animals can be readily used to conduct assays in C. elegans.

In another embodiment, the animal is a fish or amphibian. Exemplary amphibians include frog (e.g., species of Xenopus) and salamanders (e.g., species of Axolotls).

In another embodiment, the animal is a flatworm. Exemplary flatworms are the free-living (e.g., non-parasitic) flatworm planaria. Planaria are in the phylum Platylhelmenthes and the class Turbellaria. There are numerous species of planaria, any of which can be readily used.

In another embodiment, the organism is a mammal such as a mouse, rat, rabbit, pig, cow, dog, cat, non-human primate, or human.

The invention contemplates the use of any of the foregoing animals. Each of these has numerous characteristics that make them suitable for particular assays or for particular methods of promoting/inhibiting proliferation, differentiation, and/or migration. The appropriate model organism can be readily selected based on the particular assays being conducted, as well as space and resource constraints. Furthermore, the appropriate developmental stage can be readily selected. Exemplary developmental stages include, but are not limited to, embryonic stages, fetal stages, tadpole stages, larval stages, juvenile stages, and adult stages. In certain embodiments, the animal is chosen due to its optical accessibility. Furthermore, the invention contemplates studying whole animals, animal fragments, or animals inflicted with an injury. An exemplary animal fragment is a bisected or trisected organism. In one embodiment, the animal fragment is a bisected or trisected planarian. Additionally, the invention contemplates the use of wild type or mutant animals. In one embodiment, the animal is a wild type embryonic, tadpole, larval, fetal, juvenile, or adult stage animal. In another embodiment, the animal is a mutant embryonic, tadpole, larval, fetal, juvenile, or adult stage animal.

In certain embodiments, it may be desirable to conduct an assay, for example an assay to identify and/or characterize a compound that modulates a particular developmental or regenerative process, in a relatively simple system. Identified compounds or candidate ion transporter proteins can later be analyzed in higher organisms including mice, rats, non-human primates, and humans.

In certain other embodiments, it may be desirable to conduct an assay in parallel using different populations of cells. For example, screening assays can be conducted in parallel using cells derived from or resident in different organisms. Alternatively, screening assays can be conducted in parallel using cells of varying developmental stages derived from or resident in the same organism. In still another embodiment, screening assays can be conducted in parallel using cells of different developmental lineages (e.g., different cell or tissue types) derived from or resident in the same model organism. In this embodiment, the cells of differing developmental lineages can be of the same or varying developmental stages.

Depending on the particular model system and biological process chosen (e.g., organism, cell type, developmental stage, etc) for study or manipulation, one of skill in the art can select the appropriate culture conditions and methods for monitoring changes in the model system. For example, certain phenotype changes can be observed and monitored based on visual inspection with either the aided or unaided eye. Other phenotypic changes can be observed using molecular, cell biological, or biophysical reagents available in the art. For example, changes in the expression of one or more molecular markers can be assessed using known techniques including, but not limited to, RT-PCR, in situ hybridization, Northern blot analysis, Western blot analysis, immunocytochemistry, immunohistochemistry, and GeneChip analysis. Further tools including, but not limited to, method of detecting changes in cell proliferation, cell death, cell survival, membrane potential, intracellular pH, ion flux and the like can also be used to detect and assess phenotypic changes in cells or organisms.

### VI. Exemplary Disease and Injuries

As outlined above, the present invention provides methods for identifying instructor cells as well as ion transporter proteins and classes of ion transporter proteins endogenously expressed in instructor cells that mediate ion flux and/or membrane potential. The present invention also provides methods for altering the membrane potential of instructor cells to ultimately control the fate of cells on which instructor cells act. Identified transporters, as well as compounds that modulate (e.g., inhibit or promote) the activity of those transporters may be useful for modulating a biological process in vitro or in vivo. In certain embodiments, one or more ion transporter proteins or compounds that modulate the expression and/or activity of ion transporter proteins may be useful in modulating proliferation, differentiation, and/or migration of progenitor cells, such as cells in culture.

Compounds, and pharmaceutical preparations thereof, that modulate the membrane potential of instructor cells or progenitor cells to induce proliferation, differentiation, and/or migration of cells may be useful in the treatment of injury or degenerative disease. Such compounds can be administered to a human or non-human patient in need of augmenting a regenerative response to disease or injury. Briefly, compounds that promote regeneration may be administered to promote the combination of proliferation, differentiation, and/or migration of cells needed to regenerate damaged, diseased, or injured tissue.

Alternatively, compounds, and pharmaceutical preparations thereof, that modulate the membrane potential of instructor cells or subject cells (e.g., progenitor cells or cancerous cells) to inhibit proliferation, differentiation, and/or migration of cells may be useful in the treatment of over-proliferative disease (e.g., neoplastic conditions).

The invention contemplates the use of compounds individually or in combination. Suitable combinations include combinations of multiple compounds identified as promoting proliferation, differentiation, and/or migration of cells by modulating ion flux and/or membrane potential of instructor cells or progenitor cells. Suitable combinations also include a compound that promotes proliferation, differentiation, and/or migration by modulating ion flux and/or membrane potential along with one or more agents conventionally used in the treatment of the particular injury or degenerative disease.

Multiple agents may act additively or synergistically, and include combinations of agents that may show little or no effect when administered alone. Furthermore, the invention contemplates the use of agents in combination with known factors that influence proliferation, differentiation, migration, or survival of a particular cell type. Still further, the invention contemplates the use of agents as part of a therapeutic regimen along with other surgical, radiological, chemical, homeopathic, or pharmacologic intervention appropriate for the particular cell type, disease or condition.

Agents which possess one of more of these characteristics may be useful in a therapeutic context. For example, injuries and diseases of the central and peripheral nervous system effect a tremendous number of people and exact a large financial and person toll. Injuries include traumatic injuries (i.e., breaks, blunt injury, burns, lacerations) to the brain or spinal cord, as well as other injuries to any region of the CNS or PNS including, but not limited to, injuries caused by bacterial infection, viral infection, cell damage following surgery, exposure to a toxic agent, cellular damage caused by cancer or other proliferative disorder, ischemia, hypoxia, and the like. Currently, effective treatments for injuries of the CNS and PNS are limited, and individuals often experience long-term deficits consistent with the extent of injury, the location of the injury, and the types of cell that are effected.

In addition to injuries of the CNS and PNS, there are a wide variety of neurodegenerative diseases that effect particular regions and/or cell types of the CNS or PNS. These diseases are often progressive in nature, and individuals afflicted with many of these diseases have few treatment options at there disposal. Exemplary neurodegenerative diseases include, but are not limited to, Parkinson's disease, Huntington's disease, Alzheimer's disease, ALS, multiple sclerosis, stroke, macular degeneration, peripheral neuropathy, and diabetic neuropathy.

In certain embodiments, compounds can be administered to promote regeneration of mesodermal or endodermal cell and tissue types. Injuries and diseases of tissues derived from the mesoderm or endoderm include, but are not limited to, myocardial infarction, osteoarthritis, rheumatoid arthritis, diabetes, cirrohsis, polycystic kidney disease, inflammatory bowel disease, pancreatitis, Crohn's disease, cancer of any mesodermal or endodermal tissue (e.g, pancreatic cancer, Wilms tumor, soft cell carcinoma, bone cancer, breast cancer, prostate cancer, ovarian cancer, uterine cancer, liver cancer, colon cancer, etc), and injuries to any mesodermal or endodermal tissue including breaks, tears, bruises, lacerations, burns, toxicity, bacterial infection, and viral infection.

Furthermore, agents identified by the methods of the present invention may be used to modulate cells of the blood and blood vessels. Exemplary agents can be used to modulate (promote or inhibit) angiogenesis. Inhibition of angiogenesis is of particular use in the treatment of many forms of cancers, as well as in conditions aggravated by excess angiogenesis such as macular degeneration. Promotion of angiogenesis is of particular use in the treatment of conditions caused or aggravated by decreased blood flow. Exemplary conditions include, but are not limited to, myocardial infarction, stroke, and ischemia. Additionally, agents identified by the methods of the present invention can be used to promote proliferation and differentiation of various cell types of the blood and can be used in the treatment of anemia, leukemia, and various immunodeficiencies.

For any of the foregoing, the application contemplates that agents may be administered alone, or may be administered in combination with other agents. Further, the application contemplates that agents identified according to the subject methods can be administered as part of a therapeutic regimen along with other treatments appropriate for the particular injury or disease being treated. For example, in the case of Parkinson's disease, a subject agent may be administered in combination with L-dopa or other Parkinson's disease medications, or in combination with a cell based neuronal transplantation therapy for Parkinson's disease. In the case of an injury to the brain or spinal cord, a subject agent may be administered in combination with physical therapy, hydrotherapy, massage therapy, and the like. In the case of peripheral neuropathy, as for example diabetic neuropathy, a subject agent may be administered in combination with insulin. In the case of myocardial infarction, the subject agent may be administered along with angioplasty, surgery, blood pressure medication, and/or as part of an exercise and diet regimen.

Physical injuries may result in cellular damage that ultimately limits the function of a particular cell or tissue. For example, physical injuries to cells in the CNS may limit the function of cells in the brain, spinal cord, or eye. Examples of physical injuries include, but are not limited to, crushing or severing of neuronal tissue, such as may occur following a fall, car accident, gun shot or stabbing wound, etc. Further examples of physical injuries include those caused by extremes in temperature such as burning, freezing, or exposure to rapid and large temperature shifts.

Physical injuries to mesodermal cell types include injuries to skeletal muscle, cardiac muscle, tendon, ligament, cartilage, bone, and the like. Examples of physical injuries include, but are not limited to, crushing, severing, breaking, bruising, and tearing of muscle tissue, bone or cartilage such as may occur following a fall, car accident, gun shot or stabbing wound, etc. Further examples of physical injuries include breaking, tearing, or bruising of muscle tissue, bone, cartilage, ligament, or tendon as may occur following a sports injury or due to aging. Further examples of physical injuries include those caused by extremes in temperature such as burning, freezing, or exposure to rapid and large temperature shifts.

Physical injuries to endodermal cell types include injuries to hepatocytes and pancreatic cell types. Examples of physical injuries include, but are not limited to, crushing, severing, and bruising, such as may occur following a fall, car accident, gun shot or stabbing wound, etc. Further examples of physical injuries include those caused by extremes in temperature such as burning, freezing, or exposure to rapid and large temperature shifts.

Further examples of an injury to any of the aforementioned cell types include those caused by infection such as by a bacterial or viral infection. Examples of bacterial or viral infections include, but are not limited to, meningitis, staph, HIV, hepatitis A, hepatitis B, hepatitis C, syphilis, human pappiloma virus, strep, etc. However, one of skill in the art will recognize that many different types of bacteria or viruses may infect cells and cause injury.

Additionally, injury to a particular cell type may occur as a consequence or side effect of other treatments being used to relieve some condition in an individual. For example, cancer treatments (chemotherapy, radiation therapy, surgery) may cause significant damage to both cancerous and healthy cells. Surgery; implantation of intraluminal devices; the placement of implants, pacemakers, shunts; and the like can all result in cellular damage.

A wide range of neurodegenerative diseases cause extensive cell damage (i.e., injury) to cells of the CNS and PNS. Accordingly, neurodegenerative diseases are candidates for treatment using the described agents. Administration of a subject agent can promote neuronal regeneration in the CNS or PNS of a patient with a neurodegenerative disease, and the promotion of neuronal regeneration can ameliorate, at least in part, symptoms of the disease. Agents may be administered individually, in combination with other agents of the invention, or as part of a treatment regimen appropriate for the specific condition being treated. The following are illustrative examples of neurodegenerative conditions which can be treated using the subject agents.

Parkinson's disease is the result of the destruction of dopamine-producing neurons of the substantia nigra, and results in the degeneration of axons in the caudate nucleus and the putamen degenerate. Although therapies such as L-dopa exist to try to ameliorate the symptoms of Parkinson's disease, to date we are unaware of treatments which either prevent the degeneration of axons and/or increase neuronal regeneration. Administration of agents with promote neuronal regeneration can help to ameliorate at least certain symptoms of Parkinson's disease including rigidity, tremor, bradykinesia, poor balance and walking problems.

Alzheimer's disease, a debilitating disease characterized by amyloid plaques and neurofibrillary tangles, results in a loss of nerve cells in areas of the brain that are vital to memory and other mental abilities. There also are lower levels of chemicals in the brain that carry complex messages back and forth between nerve cells. Alzheimer's disease disrupts normal thinking and memory. The incidence of Alzheimer's disease will only increase as the average life expectancy continues to rise around the world. One of the most notable features of Alzheimer's disease is that affected individuals can live for extended periods of time (ten or more years) while being in an extremely debilitated state often requiring round the clock care. Accordingly, the disease takes not only an enormous emotional toll, but also exacts a tremendous financial toll on affected individuals and their families. Therapies which improve neuronal function have substantial utility in improving the quality of life of Alzheimer's sufferers.

Huntington's disease is a degenerative disease whose symptoms are caused by the loss of cells in a part of the brain called the basal ganglia. This cell damage affects cognitive ability (thinking, judgment, memory), movement, and emotional control. Symptoms appear gradually, usually in midlife, between the ages of 30 and 50. However, the disease can also strike young children and the elderly. Huntington's disease is a genetic disorder. Although people diagnosed with the disease can often maintain their independence for several years following diagnosis, the disease is degenerative and eventually fatal. Currently, there are no treatments available to either cure or to ameliorate the symptoms of this disease. Furthermore, the onset of Huntington's disease is typically in middle-age (approx age 40), at a time when many people have already had children. Thus, people have usually passed this fatal genetic disorder to their off-spring before they realize that they are ill.

Amyotrophic lateral sclerosis (ALS), often referred to as “Lou Gehrig's disease,” is a progressive neurodegenerative disease that attacks motor nerve cells in the brain and the spinal cord. Degeneration of motor neurons affect the ability of the brain to initiate and control muscle movement. With all voluntary muscle action affected, patients in the later stages of the disease become totally paralyzed, and eventually die.

Multiple sclerosis (MS) is an illness diagnosed in over 350,000 persons in the United States today. MS is characterized by the appearance of more than one (multiple) areas of inflammation and scarring of the myelin in the brain and spinal cord. Thus, a person with MS experiences varying degrees of neurological impairment depending on the location and extent of the scarring. The most common characteristics of MS include fatigue, weakness, spasticity, balance problems, bladder and bowel problems, numbness, vision loss, tremor and vertigo. The specific symptoms, as well as the severity of these symptoms, varies from patient to patient and is largely determined by the particular location within the brain of the lesions.

MS is considered an autoimmune disease. Recent data suggest that common viruses may play a role in the onset of MS. If so, MS may be caused by a persistent viral infection or alternatively, by an immune process initiated by a transient viral infection in the central nervous system or elsewhere in the body. Epidemiological studies indicating the distribution of MS patients suggest that there is a triggering factor responsible for initiating onset of the disease. Without being bound by theory, it appears that some environmental factor, most likely infectious, must be encountered.

The incidence of MS is higher in North America and Europe and this geographic distribution is further suggestive of an environmental influence(s) underlying onset of MS. Additionally, MS is more prevalent in women than in men, and is more common amongst Caucasians than within either Hispanic or African-American populations. Interestingly, MS is extremely rare within Asian populations.

Macular degeneration is a catch-all term for a number of different disorders that have a common end result: the light-sensing cells of the central region of the retina—the macula—malfunction and eventually die, with gradual decline and loss of central vision, while peripheral vision is retained. Most cases of macular degeneration are isolated, individual, occurrences, mostly in people over age 60. These types are called Age Related Macular Degeneration (AMD). More rarely however, younger people, including infants and young children, develop macular degeneration, and they do so in clusters within families. These types of macular degeneration are collectively called Juvenile Macular Degeneration and include Stargardt's disease, Best's vitelliform macular dystrophy, Doyne's honeycomb retinal dystrophy, Sorsby's fundus dystrophy, Malattia levintinese, Fundus flavimaculatus, and Autosomal dominant hemorrhagic macular dystrophy.

The present invention makes available effective therapeutic agents for restoring cartilage function to a connective tissue. Such methods are useful in, for example, the repair of defects or lesions in cartilage tissue which is the result of degenerative wear such as that which results in arthritis, as well as other mechanical derangements which may be caused by trauma to the tissue, such as a displacement of torn meniscus tissue, meniscectomy, a laxation of a joint by a torn ligament, misalignment of joints, bone fracture, or by hereditary disease. The present reparative method is also useful for remodeling cartilage matrix, such as in plastic or reconstructive surgery, as well as periodontal surgery. The present method may also be applied to improving a previous reparative procedure, for example, following surgical repair of a meniscus, ligament, or cartilage. Furthermore, it may prevent the onset or exacerbation of degenerative disease if applied early enough after trauma.

Such connective tissues as articular cartilage, interarticular cartilage (menisci), costal cartilage (connecting the true ribs and the sternum), ligaments, and tendons are particularly amenable to treatment. As used herein, regenerative therapies include treatment of degenerative states which have progressed to the point of which impairment of the tissue is obviously manifest, as well as preventive treatments of tissue where degeneration is in its earliest stages or imminent. The subject method can further be used to prevent the spread of mineralisation into fibrotic tissue by maintaining a constant production of new cartilage.

In an illustrative embodiment, the subject method can be used to treat cartilage of a diarthroidal joint, such as a knee, an ankle, an elbow, a hip, a wrist, a knuckle of either a finger or toe, or a temperomandibular joint. The treatment can be directed to the meniscus of the joint, to the articular cartilage of the joint, or both. To further illustrate, the subject method can be used to treat a degenerative disorder of a knee, such as which might be the result of traumatic injury (e.g., a sports injury or excessive wear) or osteoarthritis.

In still further embodiments, agents of the present invention can be employed for the generation of bone (osteogenesis) at a site in the animal where such skeletal tissue is deficient. For instance, administration of an agent that promotes the differentiation of stem cells to bone can be employed as part of a method for treating bone loss in a subject, e.g. to prevent and/or reverse osteoporosis and other osteopenic disorders, as well as to regulate bone growth and maturation. For example, preparations comprising the identified agents can be employed, for example, to induce endochondral ossification. Therapeutic compositions can be supplemented, if required, with other osteoinductive factors, such as bone growth factors (e.g. TGF-β factors, such as the bone morphogenetic factors BMP-2 and BMP-4, as well as activin), and may also include, or be administered in combination with, an inhibitor of bone resorption such as estrogen, bisphosphonate, sodium fluoride, calcitonin, or tamoxifen, or related compounds.

The present invention provides methods and compounds that can be used to promote regeneration, for example, regeneration of endodermally derived cells, tissues, and organs. The present invention also provides methods and compounds that can be used to promote generation, for example, of epithelial tissue, muscle tissue, nervous tissue, or connective tissue from various progenitor cells. Such methods and compositions can be used to treat conditions associated, in whole or in part, by loss of, injury to, or decrease in functional performance of endodermal cell types. By way of example, definitive endodermal cell type include, but are not limited to, hepatocytes of the liver, pancreatic cell types such as β-islet cells, cells of the lung, and cells of the gastrointestinal tract. The following are illustrative of disease states that can be treated using agents that promote regeneration of specific endodermal cell types.

Additionally, as described herein, compounds, and pharmaceutical preparations thereof, that modulate the membrane potential of instructor cells or subject cells (e.g., progenitor cells or cancerous cells) to inhibit proliferation, differentiation, and/or migration of cells may be useful in the treatment of over-proliferative disease (e.g., neoplastic conditions). Exemplary neoplastic conditions include, but are not limited to, melanoma, small cell lung cancer, non-small cell lung cancer, glioma, hepatocellular (liver) carcinoma, thyroid tumor, gastric (stomach) cancer, prostate cancer, breast cancer, ovarian cancer, bladder cancer, lung cancer, glioblastoma, endometrial cancer, kidney cancer, colon cancer, pancreatic cancer, esophageal carcinoma, head and neck cancers, mesothelioma, sarcomas, cholangiocarcinoma, small bowel adenocarcinoma, pediatric malignancies and epidermoid carcinoma.

Moreover, as is clear from the present disclosure, the methods of the invention can be used to modulating membrane potential of cells and tissues, in vitro or in vivo. Thus, in addition to various utilities in marking and identifying instructor cells and subject cells, as well as utilities in therapeutic methods, the present disclosure has a range of utilities for use to modulate cell behavior in vitro. By way of example, the disclosure provides methods of modulating progenitor cell behavior, as well as behavior of cancer cells, and these methods includes in vitro methods. Exemplary uses for such methods include: generating cell or tissue types of interest from progenitor cells in vitro; modeling cancer treatment or diagnosis in vitro; modeling cell or tissue regeneration in vitro; and generating tissue types of interest from cells or tissues, other than progenitor cells (e.g., such as to use in regenerative medicine).

### VII. Administration

For any of the foregoing classes of compounds that can be used in the various methods of the present invention, one of skill in the art can select amongst available delivery methods to administer the compound to the particular cells in vitro or in vivo. In a particular embodiment, compounds are administered to a patient in need of treatment for a particular disease or indication. The term administering is used to refer to providing a compound to cells or tissues either in vitro or in vivo. By way of example, many compounds readily transit epidermal barriers and other biological membranes. To administer such compounds to cells or to an animal, the compound can simply be dissolved and added to the fluid in which the cells or animal is cultured. Alternatively, the compound can be dissolved and added to the animals food or drinking water. In another alternative, the compound can be administered to the animal via local or systemic injection. In another embodiment, the compound can be administered locally via local application of a bandage comprising the compound or by immersing tissue in a bath comprising the compound.

Certain compounds do not as readily transit epidermal barriers and biological membranes, and thus additional techniques have been adapted to administer such compounds to cells, tissues, and organisms. For example, RNAi constructs are often administered to animals by addition to their food or drinking water. Numerous types of nucleic acids are delivered via viral or plasmid-based expression vectors. Polypeptide-based compounds that do not readily transit membrane or that are not actively transported into cells via receptor-mediated mechanisms can be administered along with carriers that facilitate transit into cells and tissues. The foregoing exemplary administration methods are well known in the art and can be selected based on the compounds and organisms being employed in the particular methods of use.

Whether compounds are being administered as part of an assay or as part of a method for modulating cell behavior, compounds can be administered alone or as pharmaceutical formulations. Exemplary pharmaceutical compositions are formulated for administration to cells or animals. In certain embodiments, the compound included in the pharmaceutical preparation may be active itself, or may be a prodrug, e.g., capable of being converted to an active compound in a physiological setting. In certain embodiments the subject compounds may be simply dissolved or suspended in water, for example, in sterile water. In certain embodiments, the pharmaceutical preparation is non-pyrogenic, i.e., does not elevate the body temperature of an animal.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a subject compound. Each carrier must be “acceptable” in the sense of being compatible with the other ingredients of the formulation and not injurious to the patient.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments the agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Another aspect of the present invention provides pharmaceutically acceptable compositions comprising an effective amount of one or more of the compounds described herein, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; wound dressing applied directly to a site of injury or wound; or (4) intravaginally or intrarectally, for example, as a pessary, cream or foam; or (5) opthalamic administration, for example, for administration following injury or damage to the retina; or (6) administration to the spinal cord to promote nerve regeneration via a continuous infusion that bathes the spinal cord. However, in certain embodiments the subject compounds may be simply dissolved or suspended in sterile water. In certain embodiments, the pharmaceutical preparation is non-pyrogenic, i.e., does not elevate the body temperature of a human or animal patient.

### EXEMPLIFICATION

The invention now being generally described, it will be more readily understood by reference to the following examples, which are included merely for purposes of illustration of certain aspects and embodiments of the present invention, and are not intended to limit the invention. For example, the particular experimental design disclosed herein represent exemplary tools and methods for validating proper function. As such, it will be readily apparent that any of the disclosed specific experimental plan can be substituted within the scope of the present disclosure.

### Example 1

### Effect of Ivermectin on Hyperpigmentation

### A. Ivermectin Exposure Induces Hyperpigmentation

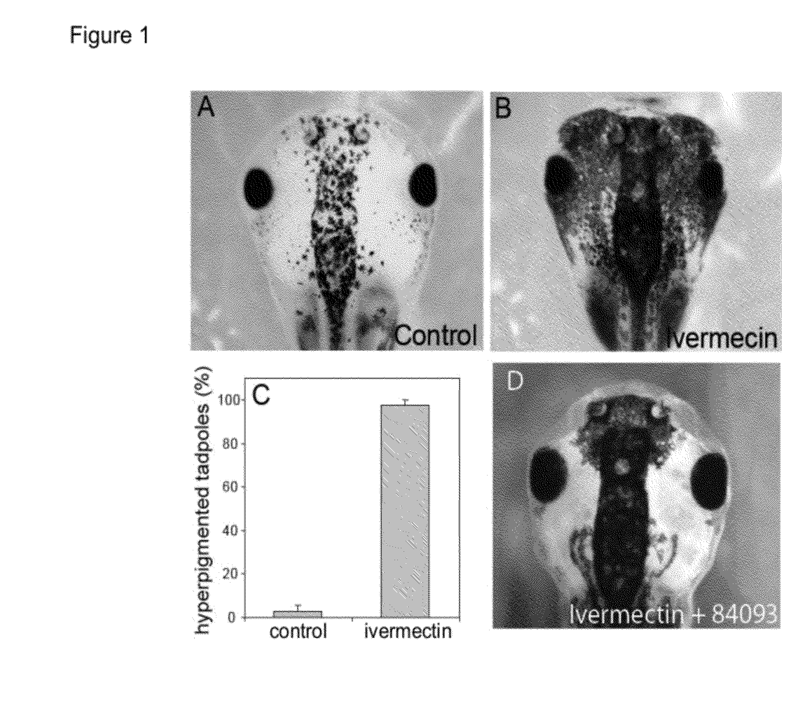

Wild type Xenopus embryos develop characteristic pigment patterns with the majority of pigment cells occupying a medial position in the head and trunk of young tadpoles (FIG. 1A). Treatment from gastrulation throughout development with ivermectin, a chloride channel activator (i.e., opens channel), resulted in a striking hyper-pigmentation in 98% of larvae (FIG. 1B, C). Melanocytes in treated individuals take on a dendritic morphology compared to wild type siblings and often migrated to regions normally devoid of pigment cells, such as the lateral eye field and base of the tail. Ivermectin-induced cell movement could be inhibited by the drug 84093, while maintaining the dendritic phenotype (FIG. 1D). Hyper-pigmenation was not accompanied by any other observable disruptions of normal development. Embryos had normal dorsoanterior index in addition to proper patterning of the body axis and organs.

### B. Hyperpigmentation is Mediated by the Glycine Receptor Chloride (GlyCl) Channel

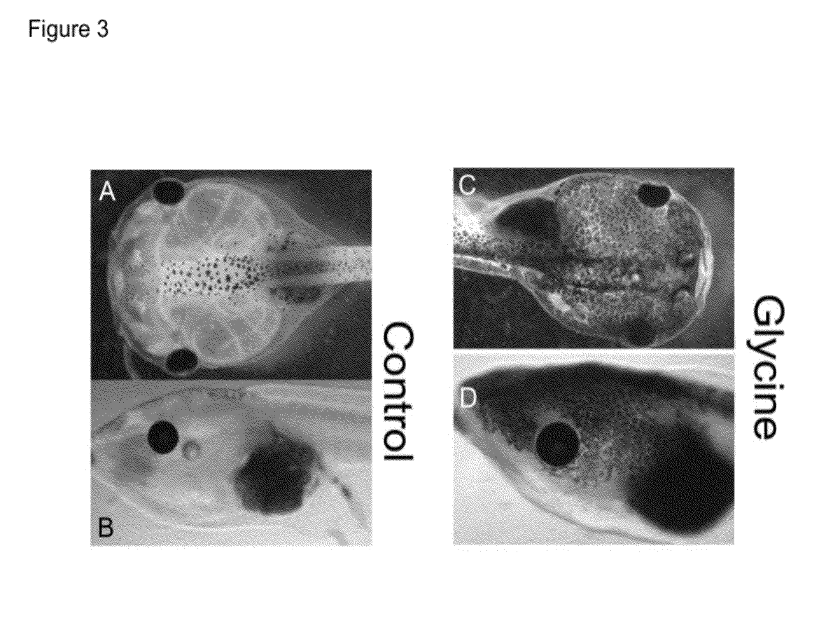

Ivermectin is known to open vertebrate GlyCl channels, resulting in chloride flux through the membrane (Schonrock, B., and Bormann, J. (1993) Naunyn Schmiedebergs Arch Pharmacol 348, 628-632; Shan, Q., Haddrill, J. L., and Lynch, J. W. (2001) J Biol Chem 276, 12556-12564.). To confirm that the hyperpigmentation effect in Xenopus embryos is likewise mediated by this receptor, we exposed embryos to the endogenous GlyCl channel ligand, glycine. Treatment with 3 mM glycine induced the same hyperpigmented phenotype as ivermectin (FIG. 3). This suggests that the modulation of pigment cell number, shape, and location is mediated by the GlyCl channel in Xenopus.

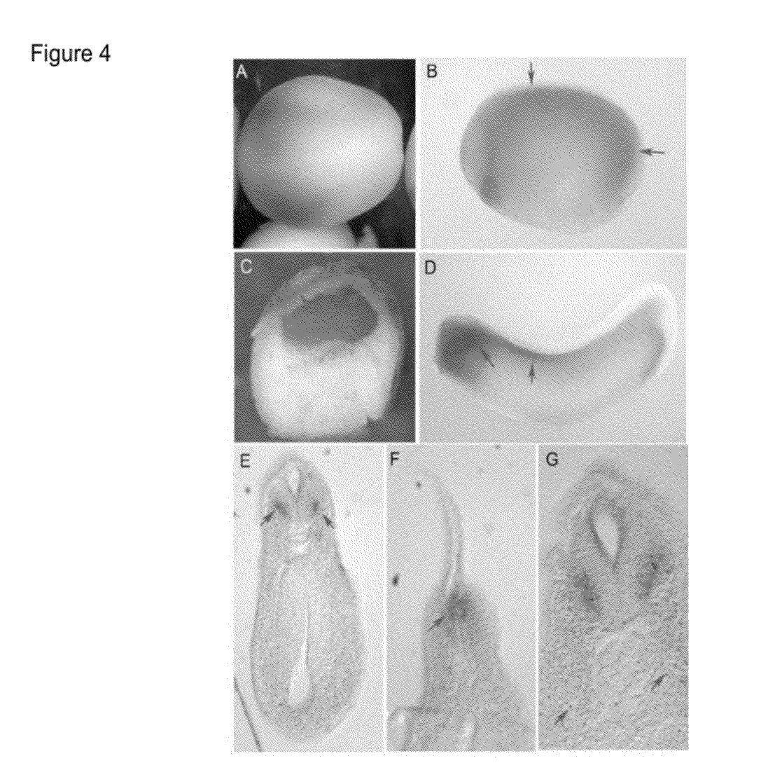

To identify the embryonic source of the hyperpigmentation signals induced by ivermectin, we asked which cells expressed the ivermectin target GlyCl. We performed in situ hybridization of embryos with an antisense probe for GlyCl subunit alpha to examine expression of the ivermectin receptor in developing embryos. Expression was first detected in early neurulating embryos across the neural plate region (FIG. 4A). Expression was stronger at stage 17 and was observed throughout the neuro-ectoderm (FIG. 4B, C; see arrows). By stage 30 expression became restricted within the central nervous system (FIG. 4D), with staining observed in the ventral neural tube in the anterior of the animal (FIG. 4E), and the dorsal neural tube in the posterior (FIG. 4F). Immunohistochemistry for the GlyCl protein in older embryo sections (stage 30+) also revealed individual punctate expression throughout the mesoderm (FIG. 4G). We conclude that the effect of ivermectin is unlikely to be only directly on the melanocytes themselves.

These results suggest that ivermectin likely acts on a population of cells described herein as instructor cells. These instructor cells express the GlyCl channel. The instructor cells non-cell-autonomously induce certain neural crest derivative cells to differentiate and spread out to take on a phenotype that differs from wild-type.

### Example 2

### Effect of Ivermectin on Level of Melanocytes

We examined whether there is a critical period of ivermectin exposure required for hyperpigmentation. Older tadpoles (beyond stage 46) exposed to ivermectin show an expansion of their pigment cells within 24 hours, but do not appear to generate more melanocytes. To demonstrate that our phenotype was indeed the result of increased melanocyte number (and not simply changes in melanocytes' shape and migration), as well as to show that ivermectin exposure is required during early development, we treated embryos with ivermectin during two different time points. One set of animals was exposed to 10 μM ivermectin from stages 10-24 (gastrulation through the completion of neurulation), while the second set were exposed from stages 28-46 (tailbud through tadpole). Early exposed embryos were washed 3× in 0.5×MMR post exposure and transferred to a new dish to remove all drug from the environment.

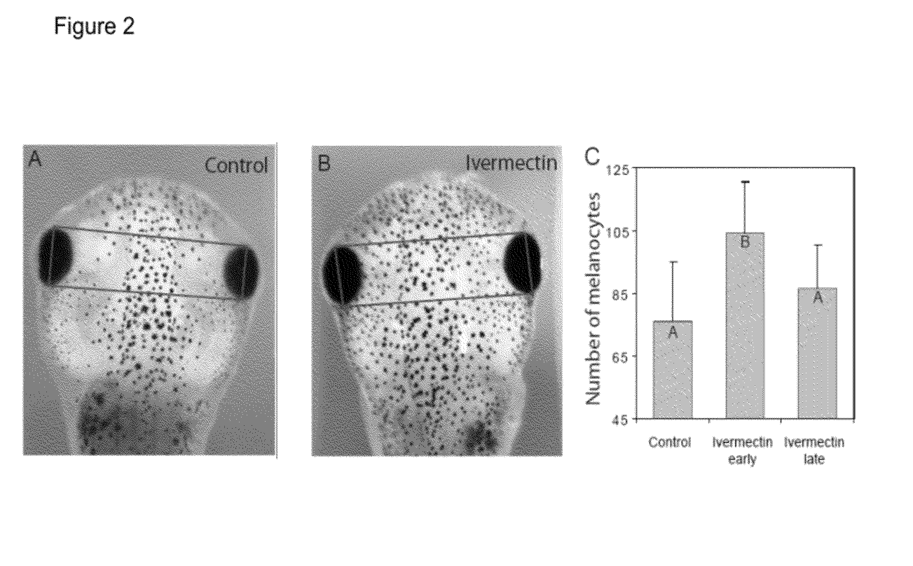

In order to evaluate the number of melanocytes between treatments, early vs. late exposed embryos were anesthetized in tricane, and then photographed. We then counted the number of melanocytes within the eye region (FIG. 2A, B), and compared each treatment to age-matched control siblings. Embryos exposed to ivermectin only late in development do not show a significant increase in melanocytes compared to control animals at stage 46 (FIG. 2C). In comparison, embryos exposed to ivermectin during gastrulation and neurogenesis, the time when a subset of neural crest cells become fated to differentiate along a melanocyte fate, show a 1.5 fold increase in melanocyte number compared to controls (FIG. 2C). These results were not confined to the eyefield, as melanocyte counts in the tip of the tail also showed a significant 1.5 fold increase when exposed to ivermectin throughout development (t test, t₄₆=6.069, p≦0.001). These results demonstrate that exposure to ivermectin late in development induces a shape change in pigment cells, but not an increase in proliferation, while early exposure induces both shape change and an increase in cell number.

### Example 3

### Effect of Membrane Voltage on Hyperpigmentation

We next sought to determine whether the alteration of melanocyte behavior was due to GlyCl-dependent changes in transmembrane potential. The intracellular concentration of chloride in an embryo is approximately 60 mM while the extracellular concentration of Modified Marc's Ringers (MMR), the solution in which the embryos are maintained, is 10 mM. If ivermectin constitutively opens chloride channels, ivermectin treatment would be expected to cause an efflux of Cl⁻ ions, thus resulting in depolarization of the cell membrane. Conversely, if the concentration of external chloride were raised above intracellular levels, chloride ions would enter cells following treatment with ivermectin, thus maintaining a hyperpolarized membrane potential. To test this hypothesis, we raised the concentration of chloride in the extracellular MMR media to 30 mM, 60 mM, or 90 mM and examined the resulting phenotype following ivermectin exposure. Raising extracellular chloride levels to 30 mM did not inhibit hyper-pigmentation (Table 2), while 60 mM suppressed and 90 mM completely inhibited the phenotype. The chloride levels tested had no observable effects on tadpole development beyond inhibiting the hyper-pigmenting effects of ivermectin.

If hyper-pigmentation is a result of membrane depolarization, then cellular voltage modulators which do not function through Glycine receptors or chloride channels should result in the same phenotype. To examine this question we examined the effect of disrupting ductin (xDuct), a H+-V-ATPase that hyperpolarizes by pumping protons across the cell membrane. Disruption of this channel was achieved by injecting YCHE78 mRNA, the dominant-negative H+-V-ATPase subunit E (dn-xDuct) into the animal pole of 1 cell embryos and examining the number of hyper-pigmented tadpoles at stage 46.

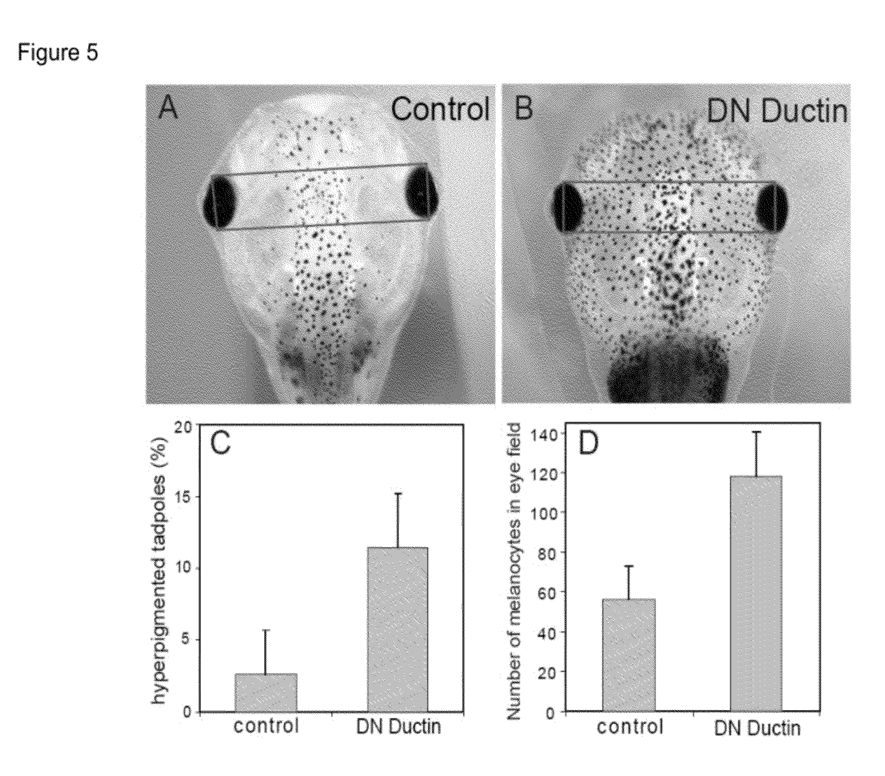

Injection of dn-xDuct induced hyper-pigmentation in 11.5 percent of embryos, significantly higher than background hyper-pigmentation observed in controls (binomial calculation, p≦0.001) (FIG. 5 A-C). As with ivermectin exposure, we wanted to demonstrate that the hyper-pigmentation was a result of an increase in melanocyte number and not simply shape change, so we counted the number of melanocytes in the eye-field of injected and un-injected embryos, as in the ivermectin treatments. We found that dn-xDuct injected hyper-pigmented embryos had 2.1× (t test, t₂₀=7.37, p≦0.001) the number of melanocytes than control animals at stage 46 (FIG. 5D).

### Example 4

### Downstream Signaling Mechanism of Ivermectin-Induced Hyper-Pigmentation

We next sought to examine how changes in membrane voltage are translated into downstream signaling. In order to determine the signaling molecules responding to ivermectin induced depolarization, we examined three known voltage sensitive pathways: calcium, serotonin, and gap junctions. We capitalized on the existence of drugs known to block each of these pathways to determine if any were downstream of ivermectin signaling. As before, Xenopus embryos were exposed to 10 μM ivermectin from gastrulation throughout development, and in addition were exposed to one of four drugs during the same period: the calcium channel inhibitors cadmium chloride and verapamil, the selective serotonin reuptake inhibitor fluoxetine, and the gap junction inhibitor lindane.

Treatment with 0.1 mM cadmium chloride, 0.1 mM verapamil, and 50 mM lindane did not result in any reduction in ivermectin-induced hyper-pigmentation (Table 3). However, exposure to 20 mM fluoxetine blocked ivermectin-induced hyper-pigmentation in all of the treated embryos. None of the drugs resulted in observable developmental defects at the doses tested, but were toxic when the concentrations were increased. These results suggest that the serotonin pathway is downstream of ivermectin signaling, and that inhibition of the serotonin pathway inhibited hyper-pigmentation.

Additionally, based on these results, promotion of a proliferative state resulting from depolarization of an instructor cell or, further, a subject cell (e.g., progenitor cell or cancerous cells) may be inhibited by a serotonin reuptake inhibitor, such as a selective serotonin reuptake inhibitor, such as fluoxetine. That is, although the present example demonstrates inhibition of an ivermectin-induced proliferative state by fluoxetine, it supports the utility of serotonin reuptake inhibitors in neoplastic conditions caused by depolarization of an instructor cell resulting from the action of a channel other than GlyCl. More specifically, the present example supports the utility of fluoxetine in the treatment of melanoma.

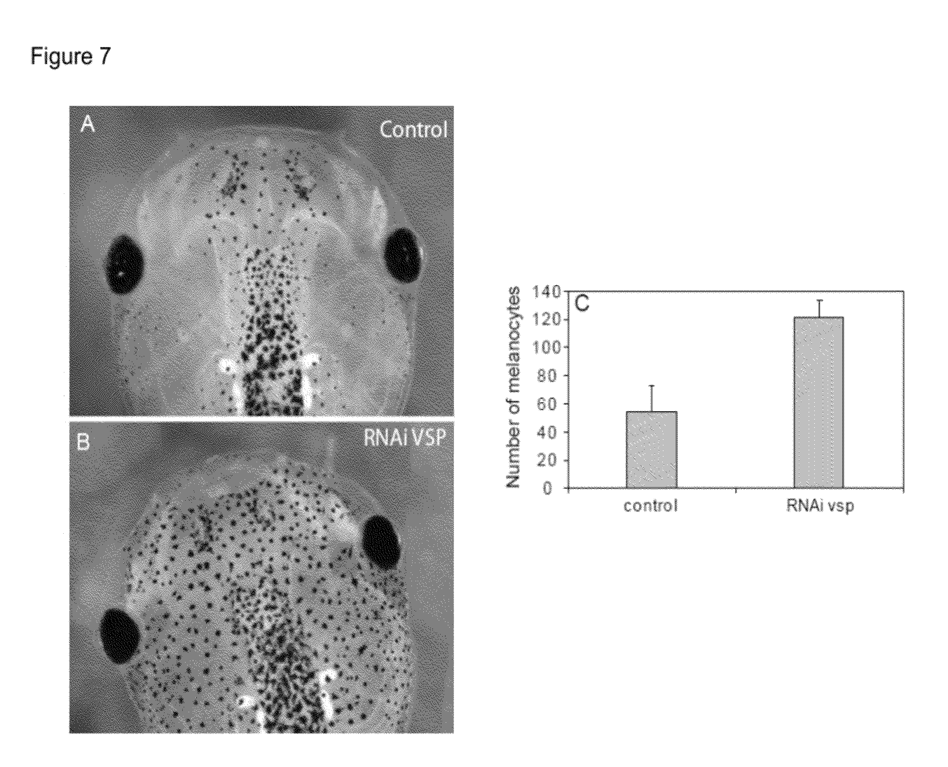

Voltage sensitive phosphatases (VSP) have also been shown to respond to modulations in membrane potential. To determine if VSP signaling is involved in hyper-pigmentation we injected a hairpin RNAi VSP construct into 1 cell embryos. Injection of the construct resulted in hyper-pigmented embryos (FIG. 7), with an excess of melanocytes compared to control tadpoles (t test, t₈=6.64, p=0.0002).

### Example 5

### Effect of Changes in Membrane Potential on Human Epidermal Melanocytes

After finding that Xenopus melanocytes responded to modulations in membrane potential, we examined human epidermal melanocytes to assess the impact of membrane depolarization on cell phenotype. For these studies, ivermectin could not be used to depolarize the membrane because the culture medium used to maintain the cells has a much higher chloride ion concentration then that observed intracellularly in these particular cells. Thus, membrane potential was modulated by raising the extracellular potassium concentration by addition of potassium gluconate to the media.

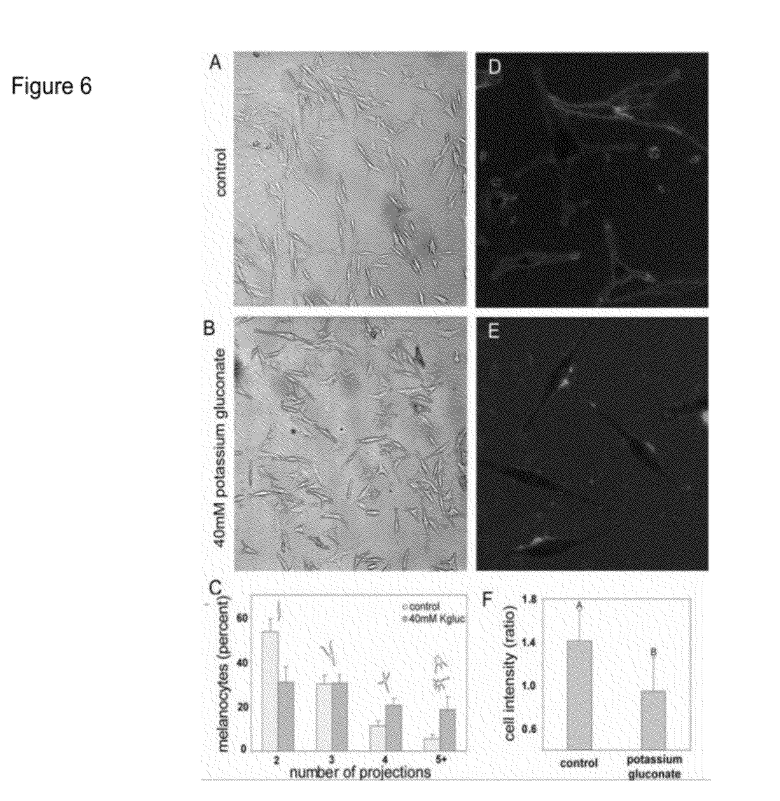

No measurable differences in cell proliferation were noted between melanocytes cultured in standard vs high potassium media. However, cells grown in high potassium media demonstrated a striking shape change similar to Xenopus melanocytes exposed to ivermectin. Following two days of culture in 50 mM potassium gluconate supplemented media, human melanocytes developed a highly denitrified morphology, with a number of cells showing five or more such projections (compare FIGS. 6a and b). When quantified, culturing cells in high potassium media had a significant effect on the number of projections of melanocytes (2way ANOVA, F₃=18.29, P≦0.001). To verify that melanocytes grown in high potassium medium were in fact depolarized we made use of membrane voltage imaging using CC2-DMPE and DiBAC₄ dyes, which allow visualization of the membrane potential (FIG. 6, d and e). Comparison of treatments showed depolarization of treated cells compared to controls (FIG. 6f, t₂₂=3.77, p=0.001).

### Example 6

### Effect of GlyCl Channel Inhibition

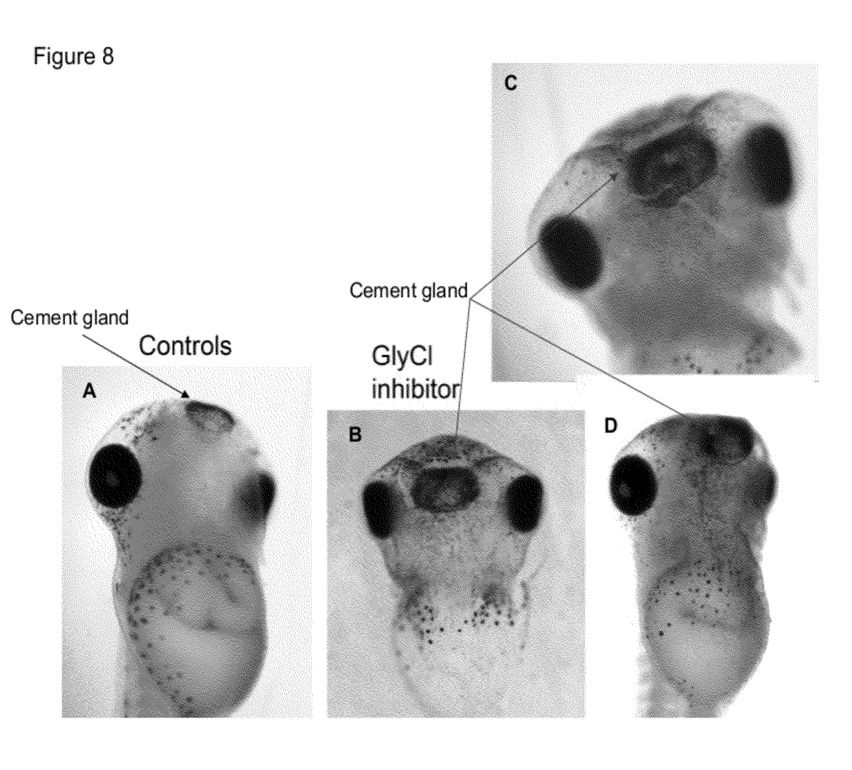

We next examined the effect of blocking the GlyCl channel on Xenopus embryos. Xenopus embryos were treated with a known GlyCl channel blocker, strychnine, at stage 14 and examined at stage 41. Notably, in contrast to opening the GlyCl with ivermectin, blocking the GlyCl channel promoted an expansion of the cement glands (a neuroectodermal derivative) of treated embryos (FIG. 8, B-D) as compared to the control embryos (FIG. 8A). It is interesting to note that cement glands are derived from neuroectodermal cells, whereas melanocytes are neural crest derivatives, suggesting that differential modulation of instructor cells (e.g., by opening a channel to cause hyperpolarization or depolarization, or blocking ion flux altogether) can have multiple phenotypic consequences that can be controlled as desired by modulating the endogenously expressed channel via an exogenously supplied small molecule or other agent.

TABLE 2

| | 10 mM | 30 mM | 60 mM | 90 mM |
|---|---|---|---|---|
| | Cl⁻ | Cl⁻ | Cl⁻ | Cl⁻ |
| Normal pigmentation | 0 | 1 | 18 | 32 |
| Hyper-pigmented | 25 | 23 | 5 | 0 |
| % hyper-pigmented | 100 | 95.8 | 21.7 | 0 |
| n | 25 | 24 | 23 | 32 |

Embryos were exposed to the chloride activator ivermectin (10 μM) from stage 10 throughout development in MMR containing varying levels of chloride. Under the two lowest levels, 10 mM and 30 mM, exposure to ivermectin resulted in strong hyper-pigmentation, with nearly all of the exposed embryos developing an excess of pigment cells over their bodies. Exposure to higher chloride levels resulted in partial inhibition of hyper-pigmentation at 60 mM concentration, and complete inhibition at 90 mM. Embryo's raised under the various chloride treatments showed no abnormal development at the conclusion of the experiment.

TABLE 3

| | | | ivermectin + | | | |
|---|---|---|---|---|---|---|
| | | | cadmium | Ivermectin + | Ivermectin + | Ivermectin + |
| | control | ivermectin | chloride | verapamil | fluoxetine | lindane |
| WT tadpoles | 57 | 3 | 1 | 0 | 38 | 0 |
| Hyper-pigmented | 0 | 61 | 47 | 52 | 0 | 48 |
| tadpoles | | | | | | |
| % hyper-pigmented | 0 | 95.3 | 97.9 | 100 | 0 | 100 |
| n | 57 | 64 | 48 | 52 | 38 | 48 |

Embryos were exposed to 10 μM ivermectin from stage 10 throughout development. Concurrently, they were also exposed to one of four drugs; the calcium channel inhibitors cadmium chloride and verapamil, the selective serotonin reuptake inhibitor fluoxetine, and the gap junction inhibitor lindane. Ivermectin alone resulted in a strong incidence of hyper-pigmentation, a phenotype absent from control embryos. Exposure to cadmium chloride, verapamil, or lindine in the presence of ivermectin did not inhibit hyper-pigmentation. However, exposure to fluoxetine completely inhibited ivermectin induced hyper-pigmentation. All drugs were used at doses that did not result in any developmental defects.
The following methods were used in conducting the experiments detailed above.

### Methods

Xenopus embryos were collected according to standard protocols (Sive, Grainger et al. 2000) in 0.1× Modified Marc's Ringers (MMR) pH 7.8+0.1% Gentamycin. Xenopus embryos were staged according to Nieuwkoop and Faber 1967.

### Expression Analysis

In situ hybridization was performed according as previously described (Harland 1991). Xenopus embryos were collected and fixed in MEMFA. Prior to in situ hybridization, embryos were washed in PBS+0.1% Tween-20 and then transferred to methanol through a 25%/50%/75% series. Probes for in situ hybridization were generated in vitro from linearized templates using DIG labeling mix from Invitrogen. Chromogenic reaction times were optimized for signal:background ratio. Marker analyses were done on 50-60 embryos for each marker.

### Microinjection

For microinjections, capped, synthetic mRNAs (Sive, Grainger et al. 2000) were dissolved in water and injected into embryos in 3% Ficoll using standard methods (50-150 msec pulses in each injected cell with borosilicate glass needles calibrated for a bubble pressure of 50-70 kPa in water). Injections delivered approximately 2.7 mL into each cell. After 3 hours embryos were washed and cultured in 0.1×MMR until desired stages.

### Drug Exposure

Stocks of ivermectin (sigma) were kept at 10 mM concentration in DMSO. Embryo exposure occurred at a 1:1000 dilution (10 μM) in MMR for the stages indicated in each experiment. Concentrations for voltage sensitive inhibitors (Sigma) were as follows: Cadmium chloride; 0.1 mM, verapamil; 0.1 mM, fluoxetine; 50 mM lindane. Stocks of strychnine were kept at 1 g in 6 ml of water; embryos were treated with 100 μl of stock in 10 ml of MMR. Drugs and media were replaced every 48 hours.

### Measurement of Melanocyte Numbers

Melanocytes were counted by cellAnalyst software (AssaySoft, Inc) on digital photographs of anesthetized larvae. Adobe Illustrator (Adobe Systems Inc) was used to define the dorsal region between the eyes used for counting. Tail regions were defined using digital photographs, starting at the most posterior tip of the tail and counting all melanocytes within a 400 pixel square bounding box extending anteriorly.

### Human Melanocyte Culture

Human melanocytes were obtained commercially and cultured in DermaLife M Melanocyte culture medium (Lifeline Cell Technology, Walkersville, Md.). Cells were maintained within standard 50 ml culture flasks (BD Bioscience, San Jose, Calif.), and were fed with 10 ml warm media every other day. Upon reaching confluence, melanocytes were passaged using a standard trypsonization protocol, and new colonies were seeded at approximately 5,000 cells per cm². For high potassium media experiments, DermaLife M media was supplemented with 40 mM potassium gluconate, a level determined to be non-inhibitory to growth while inducing morphological changes during a previous potassium dose response screen.

For dendrite analysis, cells were imaged on a Nikon AZ100M stereo microscope and the numbers of dendrites on all cells within the field of vision were counted. Cell culture in high potassium media was repeated three times, and the results averaged for statistical analysis.

### Imaging Membrane Voltage Using CC2-DMPE and DiBAC₄(3)

CC2-DMPE (N-(6-chloro-7-hydroxycoumarin-3-carbonyl)-dimyristoylphosphatidyl ethanolamine), a coumarin phospholipid, and DiBAC₄(3) (bis-(1,3-dibutylbarbituric acid) trimethine oxonol) were purchased from Invitrogen. Stock CC2-DMPE solution was prepared according to manufacturer directions; briefly, a 5 mM stock (in DMSO) was prepared, aliquoted, and stored at −20° C. until immediately before use. DiBAC₄(3) stock (1.9 mM in DMSO) was prepared and stored at room temperature. CC2-DMPE stock was dissolved 1:1000 directly into DMEM. DiBAC₄(3) stock was dissolved 1:2 in DMSO, then spun at rcf 20,800 for 10 minutes to remove undissolved particles of dye. Supernate was then diluted 1:4000 in DMEM. 1 mL of CC2-DMPE is added to cells grown in 35 mm FluoroDish Sterile Culture Dishes. Cells are incubated for 30 minutes, then washed twice with plain DMEM. 1-2 mLs of DiBAC₄(3) are then added to the dish. Cells are incubated at least 30 minutes before imaging begins; cells are imaged while in the DiBAC₄(3) bath. A round coverslip is dropped into the dish, and any medium outside the well is removed. The dish is then turned over and the cells are imaged through the glass bottom of the dish.

An Olympus BX-61 equipped with a Hamamatsu ORCA AG CCD camera, and controlled by IPLabs, was used for imaging. CC2-DMPE is imaged with the following filters: EX 405/20; BS 425; EM 460/50 (Chroma filter set 31036). DiBAC₄(3) is imaged with: EX 470/20; BS 485; EM 517/23 (Chroma filter set 41001). After darkfield (to remove camera noise) and flatfield (to correct for uneven illumination) corrections, image arithmetic is used to take the ratio CC2-DMPE intensity over DiBAC₄(3) intensity. The result is a picture of membrane voltage; the brighter the pixel, the more polarized the region it represents. Images were pseudocolored to make the contrast between different regions more easily visible. No calibration was performed; nonetheless, pixel intensity within and among images can be compared for relative quantification. Except for resizing during figure preparation, no other changes were made to the images, thus pixel intensity/color is a reliable reporter of membrane voltage.

### Statistics

All statistical analyses were performed using Prism v.5 (GraphPad software inc, La Jolla, Calif.). Student's t-tests were used for comparisons of melanoctye number, incidence of hyperpigmented tadpoles, and cell intensity for microscopy experiments. Filopodial numbers in human melanoctyes were compared using a 2way ANOVA. Data conformed to parametric requirements; no corrections were needed for normality or variance.

### REFERENCES

Adams D S, Levin M. 2006a. Inverse drug screens: a rapid and inexpensive method for implicating molecular targets. Genesis 44:530-540.
Adams D S, Levin M. 2006b. Strategies and techniques for investigation of biophysical signals in patterning. In: Whitman M, Sater A K, editors. Analysis of Growth Factor Signaling in Embryos: Taylor and Francis Books. p 177-262.
Adams D S, Masi A, Levin M. 2007. H+ pump-dependent changes in membrane voltage are an early mechanism necessary and sufficient to induce Xenopus tail regeneration. Development 134:1323-1335.
Adams D S, Robinson K R, Fukumoto T, Yuan S, Albertson R C, Yelick P, Kuo L, McSweeney M, Levin M. 2006. Early, H+-V-ATPase-dependent proton flux is necessary for consistent left-right patterning of non-mammalian vertebrates. Development 133:1657-1671.
Aw S, Adams D S, Qiu D, Levin M. 2008. H,K-ATPase protein localization and Kir4.1 function reveal concordance of three axes during early determination of left-right asymmetry. Mech Dev 125:353-372.
Chen Y, Shao J Z, Xiang L X, Dong X J, Zhang G R. 2008. Mesenchymal stem cells: a promising candidate in regenerative medicine. Int J Biochem Cell Biol 40:815-820.
Cone C D, Cone C M. 1976. Induction of mitosis in mature neurons in central nervous system by sustained depolarization. Science 192:155-158.
Cuenca-Lopez M D, Zamora-Navas P, Garcia-Herrera J M, Godino M, Lopez-Puertas J M, Guerado E, Becerra J, Andrades J A. 2008. Adult stem cells applied to tissue engineering and regenerative medicine. Cell Mol Biol (Noisy-le-grand) 54:40-51.
Darr H, Benvenisty N. 2006. Factors involved in self-renewal and pluripotency of embryonic stem cells. Handb Exp Pharmacol:1-19.
Harland, R. (1991). In situ hybridization: An improved whole mount method for Xenopus embryos. Xenopus laevis: Practical uses in cell and molecular biology. B. Kay and H. Peng. San Diego, Academic Press. 36: 685-695.
Ingber D E, Levin M. 2007. What lies at the interface of regenerative medicine and developmental biology? Development 134:2541-2547.
Levin M. 2007. Large-scale biophysics: ion flows and regeneration. Trends in Cell Biology 17:262-271.
Levin M, Thorlin T, Robinson K R, Nogi T, Mercola M. 2002. Asymmetries in H+/K+-ATPase and cell membrane potentials comprise a very early step in left-right patterning. Cell 111:77-89.
Morokuma J, Blackiston D, Adams D S, Seebohm G, Trimmer B, Levin M. 2008a. Modulation of potassium channel function confers a hyperproliferative invasive phenotype on embryonic stem cells. Proc Natl Acad Sci USA 105:16608-16613.
Morokuma J, Blackiston D, Levin M. 2008b. KCNQ1 and KCNE1 K+ channel components are involved in early left-right patterning in Xenopus laevis embryos. Cell Physiol Biochem 21:357-372.
Nieuwkoop, P. and J. Faber (1967). Normal table of Xenopus laevis (Daudin). Amsterdam, North-Holland Publishing Company.
Nogi T, Levin M. 2005. Characterization of innexin gene expression and functional roles of gap junctional communication in planarian regeneration. Dev Biol 287:314-335.
Oviedo N J, Levin M. 2007. smedinx-11 is a planarian stem cell gap junction gene required for regeneration and homeostasis. Development 134:3121-3131.
Sive, H., R. Grainger, et al. (2000). Early development of Xenopus laevis. New York, Cold Spring Harbor Laboratory Press.
Stillwell E F, Cone C M, Cone C D. 1973. Stimulation of DNA synthesis in CNS neurones by sustained deplorisation. Nature—New biology 246:110-111.
Sundelacrus S, Levin M, Kaplan D L. 2008. Membrane potential controls adipogenic and osteogenic differentiation of mesenchymal stem cells. PLoS One 3:e3737.

### INCORPORATION BY REFERENCE

All publications and patents mentioned herein, are hereby incorporated by reference in their entirety as if each individual publication or patent was specifically and individually indicated to be incorporated by reference.

1. A method of promoting tissue regeneration, comprising contacting an effective amount of a macrocyclic lactone to a cell culture, wherein the macrocyclic lactone alters the membrane potential of said cells through an endogenous ligand-gated channel expressed in said cell, thereby promoting tissue regeneraton.

2. A method for promoting tissue regeneration, comprising administering an effective amount of a macrocyclic lactone to cells, wherein the macrocyclic lactone alters the membrane potential of said cells through an endogenous ligand-gated channel expressed in said cells, thereby promoting tissue regeneraton.

3. The method of claim 1, wherein the cell culture comprises a progenitor cell.

4. The method of claim 3, wherein said progenitor cell is a neural crest progenitor cell.

5. The method claim 3, wherein said progenitor cell is a mesenchymal stem cell.

6. The method of claim 3, wherein said progenitor cell is a human mesenchymal stem cell.

7. The method of claim 3, wherein said progenitor cell is a neural stem cell or a neuroectodermal stem cell.

8. The method of claim 3, wherein said progenitor cell is an embryonic stem cell.

9. The method of claim 1, wherein said macrocyclic lactone is selected from at least one member of the group: avermectin, ivermectin, eprinomectin, abamectin, or moxidectin.

10. The method of claim 1, wherein said method or use promotes proliferation, differentiation, and/or migration of the progenitor cell.

11. The method of claim 10, wherein said progenitor cell becomes depolarized.

12. The method of claim 10, wherein said progenitor cell becomes hyperpolarized.

13. The method of claim 1, wherein said ligand-gated channel is a chloride channel.

14. The method of claim 2, wherein promoting tissue regeneration comprises promoting proliferation, differentiation, and/or migration of one or more of epithelial tissue, muscle tissue, nervous tissue, or connective tissue.

15. The method of claim 3, further comprising decreasing the extracellular chloride concentration relative to that in the progenitor cell to promote efflux of chloride ions from the progenitor cell, thereby altering the membrane potential of the progenitor cell.

16. The method of claim 3, further comprising increasing the extracellular chloride concentration relative to that in the progenitor cell to promote influx of chloride ions to the progenitor cell, thereby altering the membrane potential of the progenitor cell.

17. A method for inhibiting proliferation, differentiation and/or migration of a depolarized cell, comprising contacting cells with a macrocyclic lactone, wherein said macrocyclic lactone restores the depolarized state of the cell, thereby inhibiting proliferation, differentiation and/or migration.

18. A method for inhibiting proliferation, differentiation and/or migration of a depolarized cell, comprising contacting cells with a selective serotonin reuptake inhibitor (SSRI), wherein said SSRI restores the depolarized state of the cell, thereby inhibiting proliferation, differentiation and/or migration.

19. The method of claim 17, wherein said cell is a cancer cell, and the use is for treating cancer.

20. The method of claim 19, wherein said cancer cell is a melanoma cell.

21. The method of claim 17, wherein said cell is a neural crest derivative.

22. The method of claim 21, wherein said neural crest derivative is melanocytes or melanocyte precursors.

23. The method of claim 17, wherein said cell is a progenitor cell.

24. The method of claim 23, wherein said progenitor cell is a mesenchymal stem cell.

25. The method of claim 24, wherein said progenitor cell is a human mesenchymal stem cell.

26. The method of claim 24, wherein said progenitor cell is a neural stem cell or a neuroectodermal stem cell.

27. The method of claim 24, wherein said progenitor cell is an embryonic stem cell.

28. The method of claim 17, wherein said macrocyclic lactone is selected from at least one member of the group: avermectin, ivermectin, eprinomectin, abamectin, or moxidectin.

29-121. (canceled)
